(19) United States
(12) Patent Application Publication
(10) Pub. No.: US 2023/0002774 A1
(43) Pub. Date: Jan. 5, 2023

(54) METHODS AND COMPOSTIONS FOR INHIBITING CORONAVIRAL REPLICATION

(71) Applicant: California Institute of Technology, Pasadena, CA (US)

(72) Inventors: Kai-Wen Cheng, Pasadena, CA (US); Shan Li, Pasadena, CA (US); Feng Wang, Pasadena, CA (US); Tsui-Fen Chou, San Marino, CA (US)

(21) Appl. No.: 17/849,132

(22) Filed: Jun. 24, 2022

Related U.S. Application Data
(60) Provisional application No. 63/215,882, filed on Jun. 28, 2021.

(51) Int. Cl. C12N 15/113; C07K 16/40; A61K 38/46; A61K 31/7088; A61K 31/519; A61K 31/4439; A61K 31/517; A61K 31/403; A61K 31/538; A61K 31/496; A61P 31/14

(52) U.S. Cl. CPC C12N 15/1137; C07K 16/40; A61K 38/465; A61K 31/7088; A61K 31/519; A61K 31/4439; A61K 31/517; A61K 31/403; A61K 31/538; A61K 31/496; A61P 31/14; C12N 2310/11; C12N 2310/14; C12N 2310/531

(57) ABSTRACT

Provided herein are methods and compositions for inhibiting p97, for the treatment of a coronavirus infection in a subject, or a symptom thereof. Upon treatment, the coronavirus infection, or a symptom thereof is reduced in the subject.

### CROSS-REFERENCE TO RELATED APPLICATIONS

The present application claims the benefit of U.S. Provisional Application No. 63/215,882, filed on Jun. 28, 2021, which is hereby incorporated by reference in its entirety.

### STATEMENT REGARDING FEDERALLY SPONSORED R&D

This invention was made with government support under Grant No. NS102279 awarded by the National Institutes for Health. The government has certain rights in the invention.

### REFERENCE TO SEQUENCE LISTING

The present application is being filed along with a Sequence Listing in electronic format. The Sequence Listing is provided as file CALTE160_SEQLIST.TXT created and last modified on Jun. 20, 2022, which is 500 bytes in size. The information in the electronic format of the Sequence Listing is incorporated herein by reference in its entirety.

### FIELD

Some embodiments described herein relate generally to methods related to p97 inhibition for treatment of coronavirus infections.

### BACKGROUND

Coronaviruses (CoVs) are a family of enveloped positive-sense single-stranded RNA viruses, that are linked to respiratory and enteric disease (V'kovski, 2021). CoVs are classified under the subfamily Coronovirinae within the family Coronaviridae and the order Nidovirales. Based on genome sequences and phylogenetic relationships, members of the Coronavirinae subfamily are further divided into four groups, Alphacoronaviruses, Betacoronaviruses, Gammacoronaviruses, and Deltacoronaviruses (V'kovski, 2021). To date, seven human CoVs (HCoVs) have been identified. HCoV-229E and HCoV-NL63 belong to the Alphacoronaviruses, whereas the others belong to three Betacoronavirus lineages. HCoV-OC43 and HCoV-HKU1 belong to lineage A SARS-CoV, and SARS-CoV-2 belongs to lineage B. MERS-CoV belongs to lineage C (Liu, 2021). Unlike the four common HCoVs (229E, OC43, NL63, and HKU1), which generally cause mild to moderate upper-respiratory tract infections (common colds), SARS-CoV, SARS-CoV-2, and MERS-CoV cause severe, acute respiratory pathologies (Hu, 2021). SARS-CoV-2 is highly transmissible and pathogenic and causes coronavirus disease 2019 (COVID-19) (Hu, 2021). There is currently only one drug that is approved by the U.S. Food and Drug Administration (FDA) for the treatment of hospitalized COVID-19 patients (Kokic, 2021). This drug, remdesivir, is a nucleotide analogue prodrug that perturbs viral replication by inhibiting viral RNA-dependent RNA polymerases (RdRPs) (Emergency use Authorization (EUA) for Remdesivir). Other current therapies are primarily supportive. As such, novel therapeutic agents that can inhibit infection and virus replication are urgently needed.

Generally, CoV infection is initiated by binding of the viral spike (S) protein to its cellular receptor. These receptors are human aminopeptidase N (APN) for HCoV-229E (Yeager, 1992), angiotensin-converting enzyme 2 (ACE2) for HCoV-NL63, SARS-CoV and SARS-CoV-2 (Hofmann, 2005; Li, 2003; Hoffman, 2020), and dipeptidyl peptidase 4 (DPP4) for MERS-CoV (Raj, 2013). Host proteases cleave the S protein upon receptor binding, triggering internalization of virus into the host cell (Shang, 2020). Delivery of viral RNA can either occur after direct fusion of the viral envelope with the plasma membrane or fusion from within endosomes, depending on the virus strain and host cell type. This step leads to the release of viral nucleocapsids into the cytoplasm of host cells (Yang, 2020). After uncoating, the viral genome is immediately translated into two coterminal polyproteins (pp), pp1a and pp1ab, that can be further cleaved by viral proteases into non-structural proteins (NSPs). These NSPs form the viral replication/transcription complex (RTC), which is required for RNA replication and transcription (V'kovski, 2021). Ultimately, viral structural proteins and the RNA genome are assembled in the ER-Golgi intermediate compartment (ERGIC) to form mature virions, which are transported to the cell surface in vesicles and released via exocytosis (V'kovski, 2021). As viruses rely on host cell machinery for both replication and to spread, blocking key host factors required for these processes may provide a broad-spectrum therapeutic approach to HCoV infection.

The AAA+ ATPase p97, also known as valosin-containing protein (VCP), is a conserved and abundant eukaryotic protein that uses the energy of ATP hydrolysis to promote conformational changes in substrate proteins (Van den Boom, 2018). p97 binds ubiquitylated proteins and dissociates them from macromolecular complexes. In some cases it facilitates the downstream degradation of substrates through the ubiquitin proteasome system (UPS) (Van den Boom, 2018). p97 cooperates with various cofactors and adaptors, and is essential in diverse cellular functions. These cellular processes include ER associated degradation (ERAD) (Ballar, 2008), cell cycle regulation (Parisi, 2018), autophagy and the lysosomal function (Papadopoulos, 2017). Interestingly, these cellular processes are highly related to the host cell machinery hijacked by viruses to promote viral replication in infected cells. Proteomics analysis indicates that GRP78, an unfolded protein response (UPR) effector, is up-regulated in cells infected with SARS-CoV-2 (Bojkova, 2020). The UPR is induced by massive viral protein production in infected cells. This host cell response in turn activates ERAD to facilitate adaptation to ER stress and enhance ER protein folding capacity (Fung, 2014). In addition, Bouhaddou et al. showed that SARS-CoV-2 infection causes cell cycle arrest between the S and G2 phase (Bouhaddou, 2020). Virus-mediated cell cycle arrest is considered an essential strategy to exploit host resources and generate host cell conditions that favor viral survival and proliferation (Su, 2020). Given that p97 is a key factor in pathways that are hijacked during viral replication, p97 inhibition might interfere with SARS-CoV-2 replication, making this a plausible therapeutic target.

### SUMMARY

In accordance with some embodiments described herein, methods for p97 inhibition for treatment of coronavirus infections are provided.

Some embodiments provided herein relate to methods of improving, ameliorating or treating a viral infection. In some embodiments, the methods include identifying a subject having a viral infection, or a symptom thereof. In some embodiments, the methods include administering to the subject an effective amount of an agent that promotes inhibition of p97. In some embodiments, the viral infection is reduced after administering the agent that promotes inhibition of p97. In some embodiments, the viral infection is coronavirus infection. In some embodiments, the coronavirus infection is caused by the members of the Coronavirinae subfamily of viruses. In some embodiments, the members of Coronavirinae subfamily of viruses include HCoV-NL63, HCoV-HKU1, HCoV-229E, HCoV-OC43, severe acute respiratory syndrome coronavirus (SARS-CoV), severe acute respiratory syndrome coronavirus 2 (SARS-CoV-2) or MERS-CoV. In some embodiments, the subject having a coronavirus infection expresses or synthesizes one or more proteins involved in the cell cycle pathway differently than in normal subjects. In some embodiments, the proteins involved in the cell cycle pathway comprise components of the proteasome and the anaphase promoting complex or cyclosome (APC/C). In some embodiments, the components of the proteasome and the APC/C comprise PSMD14, PSMB3, CDC27 and CDC20. In some embodiments, the agent that promotes inhibition of p97 is an inhibitory nucleic acid molecule. In some embodiments, the inhibitory nucleic acid molecule is an antisense nucleic acid. In some embodiments, the inhibitory nucleic acid molecule is a siRNA. In some embodiments, the inhibitory nucleic acid molecule is a shRNA. In some embodiments, the inhibitory nucleic acid molecule corresponds to or is complementary to at least a fragment of nucleic acid encoding p97. In some embodiments, the agent that promotes inhibition of p97 is a p97 binding antagonist. In some embodiments, the p97 binding antagonist inhibits the binding of p97 to its partners. In some embodiments, the p97 binding antagonist is an antibody against p97 or a fragment of p97. In some embodiments, the antibody is a monoclonal, polyclonal or an antibody fragment selected from the group consisting of Fab, Fab′-SH, Fv, scFv, and (Fab′)₂ fragments. In some embodiments, the agent that promotes inhibition of p97 is a genetic tool. In some embodiments, the genetic tool is selected from the group consisting of a CRISPR/Cas9 system, a zinc finger nuclease system, a TALEN system, a homing endonucleases system or a meganuclease system. In some embodiments, the agent that promotes inhibition of p97 is a small molecule inhibitor. In some embodiments, the small molecule inhibitor that promotes inhibition of p97 is CB-5083, NMS-873, NMS-859, DBeQ, MSC1094308, ML240, p97-IN-1, VCP/p97 inhibitor-1, ML241 hydrochloride, or UPCDC-30245.

Some embodiments provided herein relate to methods of improving, ameliorating or treating a viral infection. In some embodiments, the methods include identifying a subject having a viral infection, or a symptom thereof. In some embodiments, the methods include administering to the subject an effective amount of an agent that promotes inhibition of p97. In some embodiments, the inhibition of p97 in the subject reduces or decreases the expression or levels of viral proteins in the subject. In some embodiments, the viral proteins comprise nucleoprotein (N), spike glycoprotein (S), membrane protein (M), non-structural protein 2a (NS2a), non-structural protein 4a (NS4a), replicase polyprotein 1ab (pp1ab) and protein 1 (1ORF). In some embodiments, the inhibition of p97 in the subject reduces or decreases the viral titer of coronavirus. In some embodiments, the inhibition of p97 in the subject reduces the cytopathic effects caused by the coronavirus infection. In some embodiments, the inhibition of p97 in the subject reduces or suppresses the replication of coronavirus.

Some embodiments provided herein relate to methods of identifying a subject having a coronavirus infection. In some embodiments, the methods include detecting at least one of a level of a product or expression of a gene of the subject selected from the group consisting of: CDC20, CDC27, PSMD14 and PSMB3, or a combination of two or more of the listed genes. In some embodiments, detecting a level of product or gene expressed differently in normal and subjects with coronavirus infection.

Some embodiments provided herein relate to methods of improving, ameliorating, or treating a coronavirus infection. In some embodiments, the methods include detecting the level, and/or expression of at least one or more of CDC20, CDC27, PSMD14 and PSMB3 in a subject. In some embodiments, the level, and/or expression of one or more of CDC20, CDC27, PSMD14 in the subject is compared to the level, and/or expression of one or more of CDC20, CDC27, PSMD14 in the normal subject. In some embodiments, the detection of an abnormal level and/or expression of one or more of CDC20, CDC27, PSMD14 and PSMB3 in the subject relative to the normal subject indicates the presence of a coronavirus infection in the subject. In some embodiments, an effective amount of an agent that promotes inhibition of p97 is administered to the subject with an abnormal level and/or expression of one or more of CDC20, CDC27, PSMD14 and PSMB3.

In some embodiments, the methods include identifying a subject who would benefit from inhibiting p97. In some embodiments, the methods include administering an effective amount of an agent to inhibit p97. In some embodiments, the subject is in need of p97 inhibition, and following administration of a p97 inhibiting agent, dysregulated cell cycle pathway is regulated.

In some embodiments, the agents that are used to inhibit p97 in a subject that is in need of treatment for motor neuron disease can be nucleic acid molecules, antagonists that binds and inhibits p97, small molecule inhibitors that inhibit p97 or genetic tools.

### BRIEF DESCRIPTION OF THE DRAWINGS

The foregoing and other features of the present disclosure will become more fully apparent from the following description, taken in conjunction with the accompanying drawings. Understanding that these drawings depict only some embodiments in accordance with the disclosure and are therefore, not to be considered limiting of its scope, the disclosure will be described with additional specificity and detail through use of the accompanying drawings.

FIGS. 1A-1F depict suppression of HCoV replication in H1299 cells by p97 inhibition. FIG. 1A shows cell lines infected with HCoV-229E (MOI 0.05) or HCoV-OC43 (MOI 0.01). Viral RNA in cell lysates was quantified by real-time PCR at 24 hpi. Data show RNA levels relative to GAPDH. Error bars represent SD. FIG. 1B depicts representative images of cytopathogenic effect (CPE) in the HCoV infected H1299 cells at 48 hpi. FIG. 1C illustrates the experimental set up for inducing, infecting, and harvesting cells. H1299 cells with inducible control shRNA (Ctrl shRNA) or p97 shRNA were inoculated with doxycycline (Dox) (0.5 mg/mL) for 72 h. Cells were infected with HCoV-229E (MOI 0.05) or HCoV-OC43 (MOI 0.01). After 1 h of infection, cells were washed and harvested at 0, 4, 8, or 24 hpi. Mock-infected cells were harvested at 24 hpi. FIG. 1D shows representative western blot images showing p97 expression and GAPDH (loading control) expression in cell lysates across the infection time course. FIG. 1E shows quantification of viral RNA in cell lysates over time after infection. Data shows RNA levels relative to GAPDH. FIG. 1F depicts the quantification of viral titer in culture media collected from HCoV-infected cells at 24 hpi by determining TCID50 in H1299 cells. Error bars represent SD. All experiments were performed in biological triplicate. Statistical analysis was conducted with Student's t test (*, P<0.05; **, P<0.01; ***, P<0.001).

FIGS. 2A-2D depict changes in cellular pathways after infection by HCoV-229E, HCoV-OC43, or SARS-CoV-2 by proteomic analysis of H1299 cells with control shRNA in response to HCoV-229E or HCoV-OC43 infection. FIG. 2A illustrates volcano plots of the change in total protein levels comparing infected cells 24 hpi vs mock infection. Up- (red) and down-regulated proteins (blue) with |log2 FC|>0.3 and −log10 P value>1.3 are highlighted and detected viral proteins are labeled. FIG. 2B shows protein abundance of all detected viral proteins across the infection time course. FIG. 2C shows an overlap analysis of proteins that were increased after HCoV infection. Proteome data from Caco-2 cells infected with SARS-CoV-2 was obtained from a previous study (Bojkova, 2020). FIG. 2D illustrates Dotplot visualization of the top 5 and shared enriched Reactome pathway terms of proteins that were increased during the 24 h period after infection in each HCoV cohort (P-adjusted value<0.05).

FIGS. 3A-3F illustrate the impact of p97 depletion on host cell cycle regulators during HCoV infection by proteomic analysis of H1299 cells with control shRNA (Ctrl shRNA) or p97 shRNA after HCoV-229E or HCoV-OC43 infection. FIG. 3A illustrates the abundance of detected viral proteins in HCoV-infected cells at 24 hpi. FIG. 3B shows representative immunoblot images for viral N protein and the loading control GAPDH in cell lysates across the infection time course. FIG. 3C shows the number of significantly regulated DEPs(|log2 FC|>0.3, P<0.05) compared between cells with control shRNA and cells with p97 shRNA over time after infection. FIG. 3D illustrates multi-list Reactome enrichment of DEPs at each time point after HCoV-229E or HCoV-OC43 infection (P-adjusted value<0.05). FIG. 3E shows relative protein levels for the common proteins identified at all time points in cell cycle pathway in response to HCoV-OC43 infection. Heatmaps show log2 fold changes of protein abundance in infected vs mock infected cells. FIG. 3F shows relative protein levels of two proteasome-related proteins, PSMD14 and PSMB3, and two components of the APC/C, CDC27 and CDC20, in cells infected with HCoV-OC43 compared with the corresponding mock-infected cells.

FIGS. 4A-4B show that p97 is essential for early stages of HCoV replication. FIG. 4A shows that H1299 cells were continuously treated with DMSO or p97 inhibitors (CB-5083 and NMS-873) at the indicated period from 30 min before infection to 8 hpi and harvested to determine viral RNA levels. All conditions were performed in biological triplicates. FIG. 4B illustrates that cells were infected with HCoV-229E (MOI 0.5) or HCoV-OC43 (MOI 0.01). Quantification of viral RNA in the cell lysates. Data show relative RNA levels to GAPDH. Error bars represent SD. Statistical analysis was conducted with Student's t test compared to group 1: *, P<0.05; a, P<0.0001; ns, no significance.

FIGS. 5A-5D depict suppression of HCoV replication by CB-5083 in H1299 cells. H1299 cells were infected with HCoV-229 (MOI 0.5), HCoV-OC43 (0.01) or mock-treated in the presence of CB5083 at the indicated concentration. Cultured media containing CB-5083 was removed at 8 hpi and replaced with fresh media without CB-5083. FIG. 5A shows that cells were fixed at 8 hpi and stained for dsRNA expression (green). Cell nuclei were counterstained with Hoechst (blue). Scale bar, 500 mM. FIG. 5B shows that cells were harvested to quantify viral RNA levels in cell lysates at 24 hpi. Data shows viral RNA levels relative to GAPDH. FIG. 5C shows quantification of titer of secreted virus in culture media collected from the HCoV-infected cells at 24 hpi by determining TCID50 in H1299 cells. FIG. 5D shows determination of cell viability of infected cells at 72 hpi and normalizing to mock-infected and DMSO-treated controls. Error bars represent SD. All conditions were performed in biological triplicate. Statistical analysis was conducted with Student's t test compared to infected control (0 mM of CB-5083): *, P<0.05; **, P<0.01; ***, P<0.001; ****, P<0.0001.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

Without being limited by any theory, it is contemplated that p97 plays a crucial role of p97 in facilitating HCoV entry and replication. p97 is a key player in regulating diverse cellular functions after viral infection. It also impacts different stages of the viral life cycle for various viruses. Therefore, targeting p97 with a specific inhibitor is a potential broad-spectrum treatment for HCoV infection.

Definitions

Unless defined otherwise, technical, and scientific terms used herein have the same meaning as commonly understood when read in light of the instant disclosure by one of ordinary skill in the art to which the present disclosure belongs. For purposes of the present disclosure, the following terms are explained below.

The embodiments herein are generally disclosed using affirmative language to describe the numerous embodiments. Embodiments also include ones in which subject matter is excluded, in full or in part, such as substances or materials, method steps and conditions, protocols, or procedures.

In some embodiments, the numbers expressing quantities of ingredients, properties such as concentration, reaction conditions, and so forth, used to describe and claim certain embodiments described herein are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments are approximations, the numerical values set forth in the specific examples are reported as precisely as practicable. The numerical values presented in some embodiments may contain certain errors necessarily resulting from the standard deviation found in their respective testing measurements.

In some embodiments, the terms “a” and “an” and “the” and similar references used in the context of describing a particular embodiment described herein (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate embodiments and does not pose a limitation on the scope of the embodiments otherwise claimed. No language in the specification should be construed as indicating any non-claimed element essential to the practice of any of the embodiments described herein.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The terms “individual”, “subject”, or “patient” as used herein have their plain and ordinary meaning as understood in light of the specification, and mean a human or a non-human mammal, e.g., a dog, a cat, a mouse, a rat, a cow, a sheep, a pig, a goat, a non-human primate, or a bird, e.g., a chicken, as well as any other vertebrate or invertebrate. The term “mammal” is used in its usual biological sense. Thus, it specifically includes, but is not limited to, primates, including simians (chimpanzees, apes, monkeys) and humans, cattle, horses, sheep, goats, swine, rabbits, dogs, cats, rodents, rats, mice, guinea pigs, or the like.

Groupings of alternative elements or embodiments disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

The term “inhibit” as used herein has its plain and ordinary meaning as understood in light of the specification and may refer to the reduction or prevention of a biological activity. The reduction can be by a percentage that is, is about, is at least, is at least about, is not more than, or is not more than about, 10%, 20%, 30%, 40%, 50%, 60%, 70%, 80%, 90%, or 100%, or an amount that is within a range defined by any two of the aforementioned values. The term inhibit may not necessarily indicate a 100% inhibition. A partial inhibition may be realized.

The term “treatment” or “treating” means any administration of a compound or an agent according to the present disclosure to a subject having or susceptible to a condition or disease disclosed herein for the purpose of: 1) preventing or protecting against the disease or condition, that is, causing the clinical symptoms not to develop; 2) inhibiting the disease or condition, that is, arresting or suppressing the development of clinical symptoms; or 3) relieving the disease or condition that is causing the regression of clinical symptoms. In some embodiments, the term “treatment” or “treating” refers to relieving the disease or condition or causing the regression of clinical symptoms.

The term “effective amount” is meant as the amount of an agent required to reduce the symptoms of a disease relative to an untreated subject. The effective amount of agent(s) used to practice any of the embodiments described herein for therapeutic treatment of a coronavirus infection varies depending upon the manner of administration, the age, body weight, and general health of the subject. Ultimately, a physician or veterinarian will decide the appropriate amount and dosage regimen. Such amount is referred to as an “effective” amount.

Preferred embodiments are described herein, including the best mode known to the inventors for carrying out certain embodiments. Variations on those preferred embodiments will become apparent to those of ordinary skill in the art upon reading the foregoing description. It is contemplated that skilled artisans can employ such variations as appropriate, and embodiments can be practiced otherwise than specifically described herein. Accordingly, many embodiments include all modifications and equivalents of the subject matter recited in the claims appended hereto as permitted by applicable law. Moreover, any combination of the above-described elements in all possible variations thereof is encompassed unless otherwise indicated herein or otherwise clearly contradicted by context.

Furthermore, numerous references have been made to patents and printed publications throughout this specification. Each of the above cited references and printed publications are herein individually incorporated by reference in their entirety.

Embodiments disclosed herein are illustrative of the principles of the disclosure. Other modifications that can be employed can be within the scope of the disclosure. Thus, by way of example, but not of limitation, alternative configurations can be utilized in accordance with the teachings herein. Accordingly, embodiments are not limited to that precisely as shown and described.

### Inhibition of p97

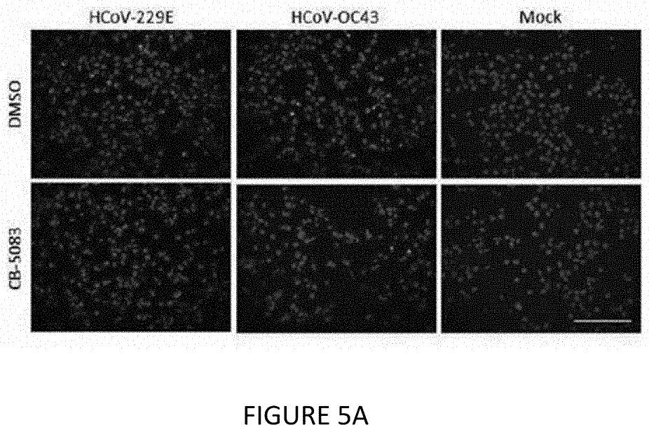

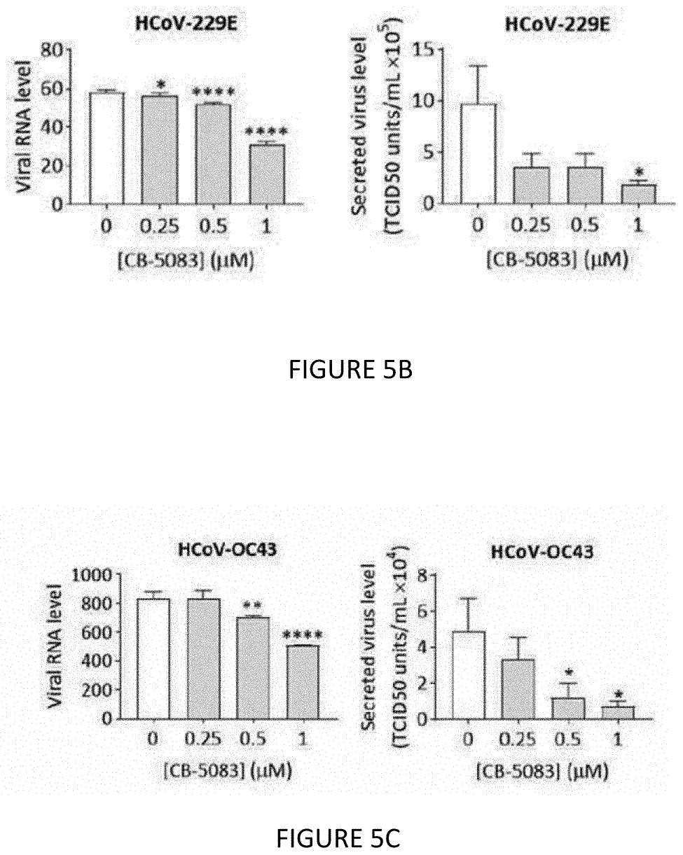

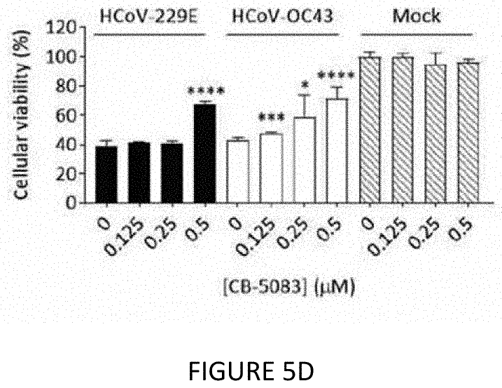

Inhibition of p97 has been observed to exhibit protective effects against coronavirus and reduce cytopathic effects induced by the virus (see FIG. 5D). Accordingly, in some embodiments described herein, methods of treatment for coronavirus infections are provided. The methods can comprise administering an effective amount of an agent that promotes inhibition of p97 in the subject with a coronavirus infection. Following administration of an agent that promotes inhibition of p97, the coronavirus infection or a symptom thereof is reduced.

Various agents can be used to inhibit p97 in a subject that is in need of treatment for coronavirus infection. For example, a nucleic acid molecule can be used to inhibit p97. In some embodiments, an antagonist that binds and inhibits p97 can be used. As another example, small molecule inhibitors that inhibit p97 can be used. As still yet another example, a genetic tool can be used to inhibit p97.

In some embodiments, inhibition of p97 reduces a coronavirus infection or a symptom thereof, wherein the coronavirus infection is caused by the members of the Coronavirinae subfamily of viruses. Non-limiting examples of Coronavirinae subfamily of viruses include HCoV-NL63, HCoV-HKU1, HCoV-229E, HCoV-OC43, severe acute respiratory syndrome coronavirus (SARS-CoV), severe acute respiratory syndrome coronavirus 2 (SARS-CoV-2) or MERS-CoV. In some embodiments, the methods include administering a therapeutically effective amount of an agent that promotes inhibition of p97 to a subject in need thereof.

In some embodiments, the subject is infected with HCoV-229E. In some embodiments, the method further comprises determining whether the subject is infected with HCoV-229E, and the effective amount of p97 inhibiting agent is administered if the subject is infected with HCoV-229E. In some embodiments, the subject is infected with HCoV-NL63. In some embodiments, the method further comprises determining whether the subject is infected with HCoV-NL63, and the effective amount of p97 inhibiting agent is administered if the subject is infected with HCoV-NL63. In some embodiments, the subject is infected with HCoV-OC43. In some embodiments, the method further comprises determining whether the subject is infected with HCoV-OC43, and the effective amount of p97 inhibiting agent is administered if the subject is infected with HCoV-OC43. In some embodiments, the subject is infected with HCoV-HKU1. In some embodiments, the method further comprises determining whether the subject is infected with HCoV-HKU1, and the effective amount of p97 inhibiting agent is administered if the subject is infected with HCoV-HKU1. In some embodiments, the subject is infected with SARS-CoV. In some embodiments, the method further comprises determining whether the subject is infected with SARS-CoV, and the effective amount of p97 inhibiting agent is administered if the subject is infected with SARS-CoV. In some embodiments, the subject is infected with SARS-CoV-2. In some embodiments, the method further comprises determining whether the subject is infected with SARS-CoV-2, and the effective amount of p97 inhibiting agent is administered if the subject is infected with SARS-CoV-2. In some embodiments, the subject is infected with MERS-CoV. In some embodiments, the method further comprises determining whether the subject is infected with MERS-CoV, and the effective amount of p97 inhibiting agent is administered if the subject is infected with MERS-CoV.

In accordance with any of the embodiments described above, an effective amount of a nucleic acid molecule that corresponds to or is complementary to at least a fragment of nucleic acid encoding p97 is administered to inhibit p97. In accordance with any of the embodiments described above, the nucleic acid molecule is a siRNA. In some embodiments, the nucleic acid molecule is a shRNA. In accordance with any of the embodiments described above, the nucleic acid molecule is an antisense nucleic acid.

In accordance with any of the embodiments described above, an effective amount of antagonist that binds and inhibits p97 is administered. In accordance with any of the embodiments described above, the antagonist is an antibody against p97 or a fragment of p97. In accordance with any of the embodiments described above, the antibody is a monoclonal, polyclonal or an antibody fragment selected from the group consisting of Fab, Fab′-SH, Fv, scFv, and (Fab′)2 fragments.

In accordance with any of the embodiments described above, a genetic tool is administered to inhibit p97. In accordance with any of the embodiments described above, the genetic tool to inhibit p97 is a CRISPR/Cas9 system. In accordance with any of the embodiments described above, the genetic tool to inhibit p97 is a zinc finger nuclease system. In accordance with any of the embodiments described above, the genetic tool to inhibit p97 is a TALEN system. In accordance with any of the embodiments described above, the genetic tool to inhibit p97 is a homing endonucleases system. In accordance with any of the embodiments described above, the genetic tool to inhibit p97 is a meganuclease system.

In accordance with any of the embodiments described above, a small molecule inhibitor is administered to inhibit p97. In accordance with any of the embodiments described above, the small molecule inhibitor to inhibit p97 is CB-5083. As used herein, the term CB-5083 has its ordinary meaning as understood in light of the specification and refers to a p97 AAA ATPase/VCP inhibitor that is orally bioavailable, and that selectively inhibits p97, and that has the chemical formula C₂₄H₂₃N₅O₂, with the chemical name of 1-(4-(benzylamino)-7,8-dihydro-5H-pyrano[4,3-d]pyrimidin-2-yl-2-methyl-1H-indole-4-carboxamide, and which has the chemical structure:

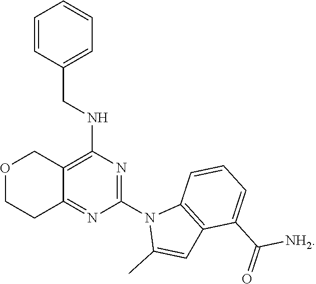 [SMILES: Cc1cc2c(C(N)=O)cccc2n1-c1nc2c(c(NCc3ccccc3)n1)COCC2]

In some embodiments, the small molecule inhibitor of p97 is CB-5083 or any functional salt, derivative, or analogue thereof.

In accordance with any of the embodiments described above, the small molecule inhibitor to inhibit p97 is NMS-873. As used herein, the term NMS-873 has its ordinary meaning as understood in light of the specification and refers to an allosteric p97 AAA ATPase/VCP inhibitor that is orally bioavailable, and that selectively inhibits p97, and that has the chemical formula C₂₇H₂₈N₄O₃S₂, with the chemical name of 3-[3-cyclopentylsulfanyl-5-[[3-methyl-4-(4-methylsulfonylphenyl)phenoxy]methyl]-1,2,4-triazol-4-yl]pyridine, and which has the chemical structure:

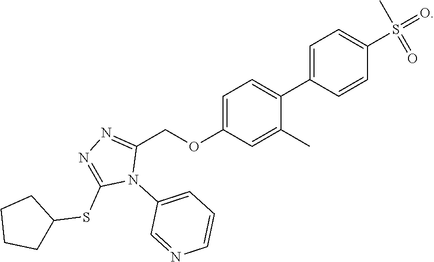 [SMILES: Cc1cc(OCc2nnc(SC3CCCC3)n2-c2cccnc2)ccc1-c1ccc(S(C)(=O)=O)cc1]

In some embodiments, the small molecule inhibitor of p97 is NMS-873 or any functional salt, derivative, or analogue thereof.

In accordance with any of the embodiments described above, the small molecule inhibitor to inhibit p97 is NMS-859. As used herein, the term NMS-859 has its ordinary meaning as understood in light of the specification and refers to a small molecule p97 AAA ATPase/VCP inhibitor, and that selectively inhibits p97, and that has the chemical formula C₁₅H₁₂ClN₃O₃S, with the chemical name of 2-chloro-N-(3-((1,1-dioxidobenzo[d]isothiazol-3-yl)amino)phenyl)acetamide, and which has the chemical structure:

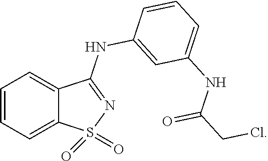 [SMILES: O=C(CCl)Nc1cccc(NC2=NS(=O)(=O)c3ccccc32)c1]

In some embodiments, the small molecule inhibitor of p97 is NMS-859 or any functional salt, derivative, or analogue thereof.

In accordance with any of the embodiments described above, the small molecule inhibitor to inhibit p97 is DBeQ. As used herein, the term DBeQ has its ordinary meaning as understood in light of the specification and refers to an ATP-competitive p97/VCP inhibitor, and that inhibits p97, and that has the chemical formula C₂₂H₂₀N₄, with the chemical name of N²,N⁴-Bis(phenylmethyl)-2,4-quinazolinediamine, and which has the chemical structure:

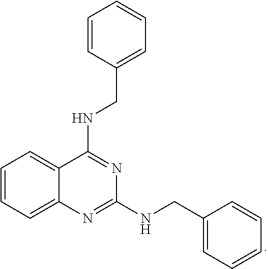 [SMILES: c1ccc(CNc2nc(NCc3ccccc3)c3ccccc3n2)cc1]

In some embodiments, the small molecule inhibitor of p97 is DBeQ or any functional salt, derivative, or analogue thereof.

In accordance with any of the embodiments described above, the small molecule inhibitor to inhibit p97 is MSC1094308. As used herein, the term MSC1094308 has its ordinary meaning as understood in light of the specification and refers to an allosteric p97 AAA ATPase/VCP inhibitor, and that inhibits p97, and that has the chemical formula C₂₉H₂₉F₃N₄, with the chemical name of N-((6-fluoro-2,3,4,9-tetrahydro-1H-carbazol-3-yl)methyl)-4,4-bis(4-fluorophenyl)butan-1-amine, and which has the chemical structure:

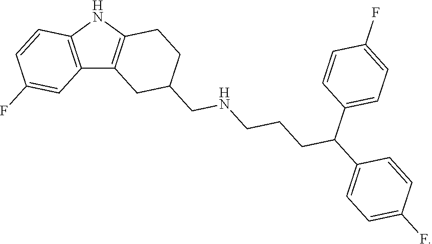 [SMILES: Fc1ccc(C(CCCNCC2CCc3[nH]c4ccc(F)cc4c3C2)c2ccc(F)cc2)cc1]

In some embodiments, the small molecule inhibitor of p97 is MSC1094308 or any functional salt, derivative, or analogue thereof.

In accordance with any of the embodiments described above, the small molecule inhibitor to inhibit p97 is ML240. As used herein, the term ML240 has its ordinary meaning as understood in light of the specification and refers to a p97 AAA ATPase/VCP inhibitor, and that selectively inhibits p97, and that has the chemical formula C₂₃H₂ON₆O, with the chemical name of 2-(2-Amino-1H-benzimidazole-1-yl)-8-methoxy-N-(phenylmethyl)-4-quinazolinamine, and which has the chemical structure:

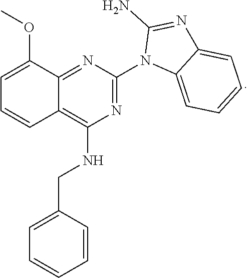 [SMILES: COc1cccc2c(NCc3ccccc3)nc(-n3c(N)nc4ccccc43)nc12]

In some embodiments, the small molecule inhibitor of p97 is ML240 or any functional salt, derivative, or analogue thereof In accordance with any of the embodiments described above, the small molecule inhibitor to inhibit p97 is p97-IN-1. As used herein, the term p97-IN-1 has its ordinary meaning as understood in light of the specification and refers to a p97/VCP inhibitor, and that selectively inhibits p97, and that has the chemical formula C₂₄H₂₄N₆O, and which has the chemical structure:

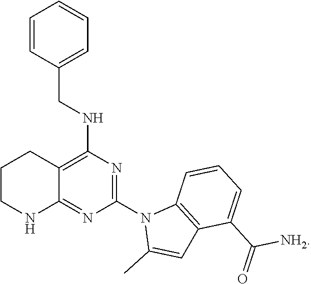 [SMILES: Cc1cc2c(C(N)=O)cccc2n1-c1nc2c(c(NCc3ccccc3)n1)CCCN2]

In some embodiments, the small molecule inhibitor of p97 is p97-IN-1 or any functional salt, derivative, or analogue thereof.

In accordance with any of the embodiments described above, the small molecule inhibitor to inhibit p97 is VCP/p97 inhibitor-1. As used herein, the term VCP/p97 inhibitor-1 has its ordinary meaning as understood in light of the specification and refers to a p97/VCP inhibitor, and that selectively inhibits p97

, and that has the chemical formula C₂₄H₂₆BN₅O₄S, and which has the chemical structure:

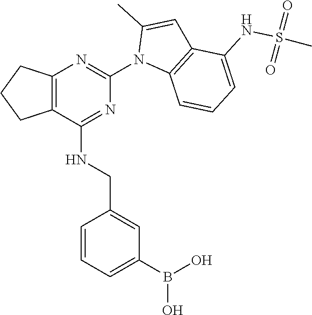 [SMILES: Cc1cc2c(NS(C)(=O)=O)cccc2n1-c1nc2c(c(NCc3cccc(B(O)O)c3)n1)CCC2]

In some embodiments, the small molecule inhibitor of p97 is VCP/p97 inhibitor-1 or any functional salt, derivative, or analogue thereof.

In accordance with any of the embodiments described above, the small molecule inhibitor to inhibit p97 is ML241 hydrochloride. As used herein, the term ML241 hydrochloride has its ordinary meaning as understood in light of the specification and refers to a p97 AAA ATPase/VCP inhibitor, and that selectively inhibits p97, and that has the chemical formula C₂₃H₂₅ClN₄O, with the chemical name of 2-(2H-benzo[b][1,4]oxazin-4(3H)-yl)-N-benzyl-5,6,7,8-tetrahydroquinazolin-4-amine hydrochloride, and which has the chemical structure:

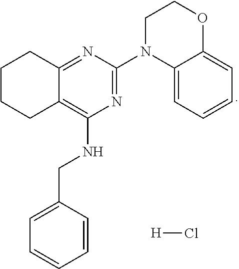 [SMILES: c1ccc(CNc2nc(N3CCOc4ccccc43)nc3c2CCCC3)cc1 ; Cl]

In some embodiments, the small molecule inhibitor of p97 is ML241 hydrochloride or any functional salt, derivative, or analogue thereof.

In accordance with any of the embodiments described above, the small molecule inhibitor to inhibit p97 is UPCDC-30245. As used herein, the term UPCDC-30245 has its ordinary meaning as understood in light of the specification and refers to an allosteric p97 AAA ATPase/VCP inhibitor, and that selectively inhibits p97, and that has the chemical formula C28H38FN5, with the chemical name of 1-(3-(5-Fluoro-1H-indol-2-yl)phenyl)-N-(2-(4-isopropylpiperazin-1-yl)ethyl)piperidin-4-amine, and which has the chemical structure:

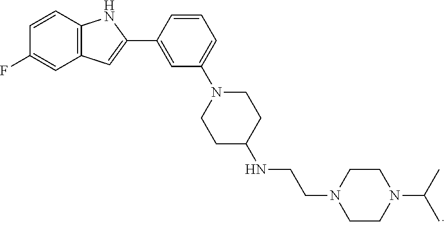 [SMILES: CC(C)N1CCN(CCNC2CCN(c3cccc(-c4cc5cc(F)ccc5[nH]4)c3)CC2)CC1]

In some embodiments, the small molecule inhibitor of p97 is UPCDC-30245 or any functional salt, derivative, or analogue thereof.

In some embodiments, inhibition of p97 decreases the synthesis, level or expression of viral proteins associated with viral entry or replication of coronavirus in host cells. Examples of viral proteins that are decreased by inhibition of p97 include, but not limited to, nucleoprotein (N), spike glycoprotein (S), membrane protein (M), non-structural protein 2a (NS2a), non-structural protein 4a (NS4a), replicase polyprotein 1ab (pp1ab) and protein 1 (1ORF).

In some embodiments, inhibition of p97 increases or decreases the synthesis, level or expression of proteins associated with regulation of cell cycle. Examples of proteins associated with regulation of cell cycle that are increased or decreased by inhibition of p97 include, but not limited to, components of proteasome and Anaphase promoting complex or cyclosome (APC/C). In some embodiments, inhibition of p97 increases or decreases the level or expression of proteasome related proteins. Examples of proteasome related proteins that are increased or decreased by depletion of p97 include, but not limited to, PSMD14 and PSMB3. In some embodiments, inhibition of p97 increases or decreases the level or expression of APC/C. Examples of APC/C related proteins that are increased or decreased by depletion of p97 include, but not limited to, CDC27 and CDC20.

In some embodiments, p97 inhibition exhibits antiviral activity during coronavirus infection. In some embodiments, inhibition of p97 reduces titer of progeny coronaviruses. In some other embodiments, inhibition of p97 prevents cytopathic effects (CPE) caused by the coronavirus infection.

As described herein, inhibiting p97 can treat, inhibit, or ameliorate coronavirus infection symptoms. As disclosed herein, amelioration is used in a broad sense to refer to at least a reduction in the magnitude of a parameter, e.g., symptom, associated with the pathological condition being treated. In some embodiments, the method can completely inhibit, e.g., prevented from happening, or stopped, e.g., terminated, such that the host no longer suffers from the pathological condition, or at least one or more of the symptoms that characterize the pathological condition. In some embodiments, the method can delay or slowing of disease progression, amelioration or palliation of the disease state, and remission (whether partial or total), whether detectable or undetectable.

### Dosage and Administration of p97 Inhibitors for Treating Coronavirus Infection

Doses of p97 inhibitors can be readily determined for a given subject based on their body mass, disease type and state, and desired aggressiveness of treatment. In some embodiments, inhibitors of p97 are administered at a dose ranging from 1 mg/kg to 200 mg/kg, such as 1, 2, 3, 4, 5, 10, 15, 20, 25, 30, 35, 40, 45, 50, 55, 60, 65, 70, 75, 80, 85, 90, 95, 100, 110, 120, 130, 140, 150, 160, 170, 180, 190, or 200 mg/kg, or an amount within a range defined by any two of the aforementioned values. The composition may be administered twice daily, once daily, twice weekly, once weekly, or once monthly, or at a frequency within a range defined by any two of the aforementioned values.

In accordance with embodiments described herein, inhibitors of p97 can be administered by any suitable route of administration. Without limitation, the inhibitors of p97 can be administered to the subject via oral administration, rectum administration, transdermal administration, intranasal administration, or inhalation. In some embodiments, the inhibitors of p97 are administered to the subject orally. In some embodiments, the inhibitors of p97 can be administered by injection or in the form of a tablet, capsule, patch or a drink.

Pharmaceutically acceptable carriers are ones which are nontoxic to the cell or mammal being exposed thereto at the dosages and concentrations employed. Pharmaceutically acceptable carriers in accordance with methods and uses and compositions herein can comprise, but not limited to, organic or inorganic, solid or liquid excipients which is suitable for the selected mode of application such as oral application or injection and administered in the form of a conventional pharmaceutical preparation, such as solid such as tablets, granules, powders, capsules, and liquid such as solution, emulsion, suspension and the like. Often the physiologically acceptable carrier is an aqueous pH buffered solution such as phosphate buffer or citrate buffer. The physiologically acceptable carrier may also comprise one or more of the following: antioxidants including ascorbic acid, low molecular weight (less than about 10 residues) polypeptides, proteins, such as serum albumin, gelatin, immunoglobulins; hydrophilic polymers such as polyvinylpyrrolidone, amino acids, carbohydrates including glucose, mannose, or dextrins, chelating agents such as EDTA, sugar alcohols such as mannitol or sorbitol, salt-forming counterions such as sodium, and nonionic surfactants such as and nonionic surfactants such as TWEEN™ surfactant, polyethylene glycol (PEG), and PLURONICS™ surfactant. Auxiliary, stabilizer, emulsifier, lubricant, binder, pH adjustor controller, isotonic agent and other conventional additives may also be added to the carriers.

### Methods of Treating a Coronavirus Infection

Described herein are methods of treatment of a coronavirus infection. In some embodiments, a subject in need of treatment for coronavirus infection is identified. In some embodiments, provided are methods for treating a coronavirus infection in a subject that is amenable to treatment by inhibiting p97.

Various methods can be used to inhibit p97 in a subject and reduce coronavirus infection, or a symptom thereof. For example, an ATP-competitor can be used to inhibit the enzyme activity of p97. In some embodiments, treatment with an allosteric p97 inhibitor can be used to inhibit p97.

In some embodiments, provided are methods for treating a coronavirus infection. In some embodiments, the coronavirus infection is caused by the members of the Coronavirinae subfamily of viruses. Non-limiting examples of Coronavirinae subfamily of viruses include HCoV-NL63, HCoV-HKU1, HCoV-229E, HCoV-OC43, severe acute respiratory syndrome coronavirus (SARS-CoV), severe acute respiratory syndrome coronavirus 2 (SARS-CoV-2) or MERS-CoV. In some embodiments, the methods include administering a therapeutically effective amount of an agent that promotes inhibition of p97, to a subject in need thereof.

In various embodiments, the method is for treating HCoV-229E infection, including inhibition of p97 in a subject who is in need of treatment for HCoV-229E infection, thereby treating the subject. In various embodiments, the method is for treating HCoV-OC43 infection, including inhibition of p97 in a subject who is in need of treatment for HCoV-OC43 infection, thereby treating the subject. In various embodiments, the method is for treating HCoV-NL63 infection, including inhibition of p97 in a subject who is in need of treatment for HCoV-NL63 infection, thereby treating the subject. In various embodiments, the method is for treating HCoV-HKU1 infection, including inhibition of p97 in a subject who is in need of treatment for HCoV-HKU1 infection, thereby treating the subject. In various embodiments, the method is for treating SARS-CoV, including inhibition of p97 in a subject who is in need of treatment for SARS-CoV infection, thereby treating the subject. In various embodiments, the method is for treating SARS-CoV-2, including inhibition of p97 in a subject who is in need of treatment for SARS-CoV-2 infection, thereby treating the subject. In various embodiments, the method is for treating MERS-CoV, including inhibition of p97 in a subject who is in need of treatment for MERS-CoV infection, thereby treating the subject.

In some embodiments as described above, the methods further comprise identifying a subject who would benefit from inhibiting p97. The methods can comprise administering an effective amount of an agent to inhibit p97. In some embodiments, the subject is in need of p97 inhibition, and following administration of p97 inhibiting agent, the coronavirus infection or a symptom thereof is reduced in the subject.

In some embodiments, the methods further comprise identifying a subject who would benefit from inhibiting p97. The methods can comprise administering an effective amount of an agent to inhibit p97. In some embodiments, the subject is in need of p97 inhibition, and following administration of a p97 inhibiting agent, dysregulated cell cycle pathway is regulated. Examples of proteins associated with regulation of cell cycle that are increased or decreased by inhibition of p97 include, but not limited to, components of proteasome and Anaphase promoting complex or cyclosome (APC/C). In some embodiments, inhibition of p97 increases or decreases the level or expression of proteasome related proteins. Examples of proteasome related proteins that are increased or decreased by depletion of p97 include, but not limited to, PSMD14 and PSMB3. In some embodiments, inhibition of p97 increases or decreases the level or expression of APC/C. Examples of APC/C related proteins that are increased or decreased by depletion of p97 include, but not limited to, CDC27 and CDC20.

In some embodiments, the methods further comprise identifying a subject who would benefit from inhibiting p97. The methods can comprise administering an effective amount of an agent to inhibit p97. In some embodiments, the subject is in need of p97 inhibition, and following administration of a p97 inhibiting agent, the synthesis, level or expression of proteins associated with viral entry or replication are regulated. Examples of viral proteins that are decreased by inhibition of p97 include, but not limited to, nucleoprotein (N), spike glycoprotein (S), membrane protein (M), non-structural protein 2a (NS2a), non-structural protein 4a (NS4a), replicase polyprotein 1ab (pp1ab) and protein 1 (1ORF).

In some embodiments, the methods further comprise identifying a subject who would benefit from inhibiting p97. The methods can comprise administering an effective amount of an agent to inhibit p97. In some embodiments, the subject is in need of p97 inhibition, and following administration of a p97 inhibiting agent, viral entry and cytopathic effects (CPE) caused by the coronavirus are reduced. In some embodiments, p97 inhibition exhibits antiviral activity during coronavirus infection. In some embodiments, inhibition of p97 reduces titer of progeny coronaviruses. In some other embodiments, inhibition of p97 prevents CPE caused by the coronavirus.

In some embodiments, the p97 inhibiting agent is administered to the subject until a coronavirus infection, or a symptom thereof in the subject is reduced. Optionally, the p97 inhibiting agent is administered to the subject after a coronavirus infection, or a symptom thereof in the subject is reduced, for example to solidify or maintain the subject free of coronavirus infections.

As described herein, inhibiting p97 can treat, inhibit, or ameliorate coronavirus infection, or a symptom thereof. As disclosed herein, amelioration is used in a broad sense to refer to at least a reduction in the magnitude of a parameter, e.g., symptom, associated with the pathological condition being treated. In some embodiments, the method can completely inhibit, e.g., prevented from happening, or stopped, e.g., terminated, such that the host no longer suffers from the pathological condition, or at least one or more of the symptoms that characterize the pathological condition. In some embodiments, the method can delay or slowing of disease progression, amelioration or palliation of the disease state, and remission (whether partial or total), whether detectable or undetectable.

### EXAMPLES

Some aspects of the embodiments discussed above are disclosed in further detail in the following examples, which are not in any way intended to limit the scope of the present disclosure.

### Experimental Material and Methods

The following experimental methods were used for Examples 1-5 described below.

Cell lines

293T, A549, H1299, MRC-5, and HCT-8 cells were purchased from ATCC. Cells were cultured in DMEM media (Sigma-Aldrich) or RPMI 1640 media (Corning) supplemented with 10% fetal bovine serum (FBS, Atlanta Biologicals), 100 mg/mL streptomycin, and 100 U/mL of penicillin (Lonza) and maintained at 37oC with 5% CO2.

### Chemical Inhibitors

p97 inhibitors, CB-5083 (Active Biochem) and NMS-873 (Xcess Biosciences Inc.) were dissolved in DMSO at concentration of 10 mM.

Generation of p97-Knockdown Cell Line

Stable H1299 cell lines expressing doxycycline-inducible shRNA against a control sequence or p97 were generated using the TripZ lent-viral shRNA system (Thermo Fisher Scientific) as described previously (Radhakrishnan, 2014). The targeted sequence for p97 is 5′-AAACAGCCATTCTCAAACAGAA-3′ (SEQ ID NO:1). The nontargeting control shRNA comes directly from Thermo Fisher Scientific. The control or p97 shRNA plasmid and lentivirus packaging plasmids (pHDM-G, CAG4-RTR2 and CAGGHIVgpco) were transiently co-transfected into 293T cells with BioT reagent (Bioland Scientific LLC). Supernatant containing the lentivirus was collected and supplemented with polybrene (8 mg/mL) to transduce H1299 cells.

### HCoV Production and Titration

The HCoV-229E strain (ATCC, VR-740) and HCoV-OC43 strain (ATCC, VR-1558) were purchased from ATCC, passaged once through MRC-5 cells (ATCC, CCL-171) and HCT-8 cells (ATCC, CCL-244), respectively, and were amplified in H1299 (ATCC, CRL-5803) or A549 cells (ATCC, CCL-185) 80 to 90% confluent cells were infected with HCoV-229E and HcoV-OC43 in a minimal volume of serum-free media for 2 h at 35° C. and 33° C. respectively. Infected cells were incubated in media containing 2% FBS for 4 to 5 days. Culture supernatants were harvested when the CPE was observed, centrifuged at 4,000 rpm for 5 min, passed through a 0.45 mm filter, and aliquoted for storage at 80° C. We measured viral titer as the median tissue culture infective dose (TCID50) per mL. H1299 cells were seeded using 5×10³ cells per well in 96-well plates in 100 mL of maintenance media. After an overnight incubation, cultured media was replaced with 100 mL fresh media. Cells were incubated with 50 mL of diluted virus stock ranging from 10-1 to 10-7-fold. After incubation at 37° C. for another 7 days, the CPE was inspected under an inverted microscope to calculate TCID50/mL.

### HCoV Infection

Cells were seeded using 1×10⁶ cells per well in 6-well plates and incubated overnight at 37° C. After three washes with serum-free media, cells were inoculated with 1 mL serum-free media containing HCoV at the indicated multiplicity of infection (MOI) for 1 hat 37° C. The 0 hpi samples were harvested immediately, while subsequent cell samples were washed three times with serum-free media before 2 mL of media with 2% FBS was added. Cells or cultured media were harvested at the indicated time points post infection.

### Detection of HCoV by Real-Time PCR

Total cell RNA was extracted using the MagMAX mirVana Total RNA Isolation Kit (Thermo Fisher Scientific) with KingFisher systems (Thermo Fisher Scientific). RNA was quantified using NanoDrop Lite Spectrophotometer (Thermo Fisher Scientific). Total RNA (1 mg) was reverse transcribed using the High-Capacity cDNA Reverse Transcription Kit (Thermo Fisher Scientific). cDNA samples were used for quantitative TagMan PCR using TaqMan™ Universal Master Mix II, no UNG (Thermo Fisher Scientific), in an QuantStudio™ 5 System (Thermo Fisher Scientific). FAM-MGB-labeled TaqMan probes for the indicated genes were purchased from Thermo Fisher Scientific. The resulting data were analyzed using Excel and GraphPad Prism 8.

### Western Blotting

Cell lysates were prepared in 50 mM HEPES pH 7.5, 200 mM NaCl, 1 mM DTT, 1% Triton-100, and protease inhibitors (Thermo Fisher Scientific). Protein concentration was determined using the Bradford protein assay (Bio-Rad). Comparable amounts (˜10 mg) of protein samples were loaded onto a 4%-20% SD S-PAGE gel (Bio-Rad) and transferred to a nitrocellulose membrane (Bio-Rad). Proteins were detected using anti-VCP antibody (1:3000) (Thermo Fisher Scientific, MA3-004) for detecting p97, anti-GAPDH antibody (1:5000) (Cell Signaling Technology, 2188S) for detecting GAPDH, anti-coronavirus antibody (1:500) (Santa Cruz Biotechnology, sc-65653) for detecting HCoV-229E nucleocapsid protein, and anti-coronavirus antibody (1:500) (Sigma-Aldrich, MAB9012) for detecting HCoV-OC43 nucleocapsid protein. Primary antibodies were detected using HRP-labeled goat anti-mouse or anti-rabbit antibodies (Bio-Rad). Blots were developed using Immobilon Western Chemiluminescent HRP Substrate (Millipore) and visualized using ChemiDoc MP Imaging System (Bio-Rad). Quantification was performed using Image Lab (Bio-Rad).

### TMT Label Proteomics

Cell lysates were prepared in 8M urea lysis buffer which was prepared by dissolving urea in the buffer containing 20 mM HEPES pH 7.5, 100 mM NaCl, protease inhibitor (Thermo Fisher Scientific) and 100 mM MG132 (Selleckchem). The protein concentration was measured by Bradford (Bio-Rad). After reduction with 10 mM TCPT (Thermo Fisher Scientific) and alkylation with 25 mM 2-chloroacetamide (Thermo Fisher Scientific), the proteins were precipitated by adding six volumes of pre-chilled acetone and incubated at −20° C. overnight. Proteins were collected by high-speed centrifugation. The dry protein pellets were resuspended in 100 mM TEAB buffer containing Lys-C (Wako Chemicals) and Trypsin (Thermo Fisher Scientific) to digest at 37° C. overnight. The peptide concentration was determined by Quantitative Fluorometric Peptide Assay (Thermo Fisher Scientific). 10mg peptide of each sample was transferred and labeled with TMTpro 16plex reagents (Thermo Fisher Scientific) by following the manufacture's instruction. 10 mg peptide of a same amount mixture of all samples was labeled with the same channel at each TMT set to be a bridge of different TMT sets. Labeled samples were combined and dried using vacuum centrifugation. Samples were then separated into 8 fractions using the High pH reversed-phase peptide Fractionation Kit (Thermo Fisher Scientific). The fractions were dissolved with 0.1% FA and peptide concentration was determined with Quantitative Colorimetric Peptide Assay (Thermo Fisher Scientific).

TMT labeled experiments were performed using an EASY-nLC 1000 connected to an Orbitrap Eclipse Tribrid mass spectrometer. 1 μg of sample was loaded onto an Aurora UHPLC Column and separated over 136 min at a flow rate of 0.4 μL/min with the following gradient: 2-6% Solvent B (7.5 min), 6-25% B (82.5 min), 25-40% B (30 min), 40-95% B (1 min), and 95% B (15 min). Solvent A consisted of 97.8% H2O, 2% ACN, and 0.2% formic acid, and solvent B consisted of 19.8% H2O, 80% ACN, and 0.2% formic acid. An MS1 scan was acquired in the Orbitrap at 120 k resolution with a scan range of 350-1800 m/z. The AGC target was 1×10⁶, and the maximum injection time was 50 ms. Dynamic exclusion was set to exclude features after 1 time for 45 s with a 5-ppm mass tolerance. MS2 scans were acquired with HCD activation type with the Orbitrap. The isolation window was 0.5 m/z, the collision energy was 32%, the maximum injection time was 86 ms and the AGC target was 5×104. Ion source settings were as follows: ion source type, NSI; spray voltage, 1650 V; ion transfer tube temperature, 300° C. System control and data collection were performed by Xcalibur software.

### Proteomic Data Processing

The proteomic analysis was performed with Proteome Discoverer 2.4 (Thermo Fisher Scientific) using the UniProt human (UP000005640), HCoV-229E (UP000006716) and HCoV-OC43 (UP000007552) databases. The SequestHT with Percolator validation. Normalization was performed relative to the total peptide amount. Volcano plots and heatmaps were generated with Prism 8. Set of proteins with significant up or down effects (Student's t test, P<0.05) in the infected samples compared with the mock control were selected and clustered by using default setting in Cluster 3.0. and TreeView. Protein clusters were increased over time after virus infection were selected for overlap analysis. Venn plots was generated with FunRich 3.1.4 (Fonseka, 2021; Pathan, 2015; Pathan, 2017). Set of proteins from each infection were tested for enrichment of Reactome pathways (Jassal, 2019) using g:Profiler (Raudvere, 2019). Significant terms were identified that have a P-adjusted value<0.05 after Benjamin-Hochberg correction. Bubble plot was generated using the R package ggplot2.

Protein abundance in infected cells with control shRNA or p97 shRNA was normalized to that in corresponding mock-infected cells. Set of proteins that were significant up- or downregulated between cells with control shRNA and p97 shRNA at each time point after infection were identified (|log2 FC|>0.3, Student's t test, P<0.05) and selected for multi-set Reactome pathway enrichment analysis using Metascape (Zhou, 2019).

### Stage-Limited Inhibition Assay

H1299 cells were treated with 0.1% DMSO, 1 mM CB-5083, or 2 mM of NMS-873 from 30 min prior to virus infection until 8 h post-infection. After infection, cells were washed three times with serum-free media and then returned to media containing 2% FBS, 0.1% DMSO, 1 mM CB-5083, or 2 mM of NMS-873. Cells were collected at 8 hpi for viral RNA determination.

### Immunostaining

Cells were fixed with cold 4% paraformaldehyde for 15 min followed by permeabilization/blocking with 10% FBS and 0.2% Triton X-100 for another 1 h. Cells were stained with mouse anti-dsRNA antibody clone rJ2 (1:50) (Sigma-Aldrich, MABE1134) followed by labeling with anti-mouse mouse secondary antibody conjugated to Alexa-488 (Thermo Fisher Scientific). Nuclei were stained with Hoechst. Fluorescent images were acquired using Thermo Scientific™ Invitrogen™ EVOS™ FL Auto 2 Imaging System.

### p97 Inhibitor Treatment

Cells were treated with the indicated concentration of CB-5083 and infected with virus. After 1 h of infection, cells were washed three times with serum-free media and then returned to media containing 2% FBS and the same concentration of CB-5083. Cultured media was replaced with fresh media at 8 hpi and harvested at 24 hpi for titration.

### Cell Viability

Cells were seeded using 2×10⁵ cells per well in 96-well plates and incubated overnight at 37° C. Cells were treated with the indicated concentration of CB-5083 and infected with virus or mock infected. After 1 h of infection, cells were washed three times with serum-free media and then cultured with media containing 10% FBS and the same concentration of CB-5083. Cultured media was replaced with fresh media at 8 hpi. At 72 hpi, cell viability was measured by CellTiter-Glo® Luminescent Cell Viability Assay (Promega).

### Example 1: p97 Facilitates HCoV Replication in Human Cells

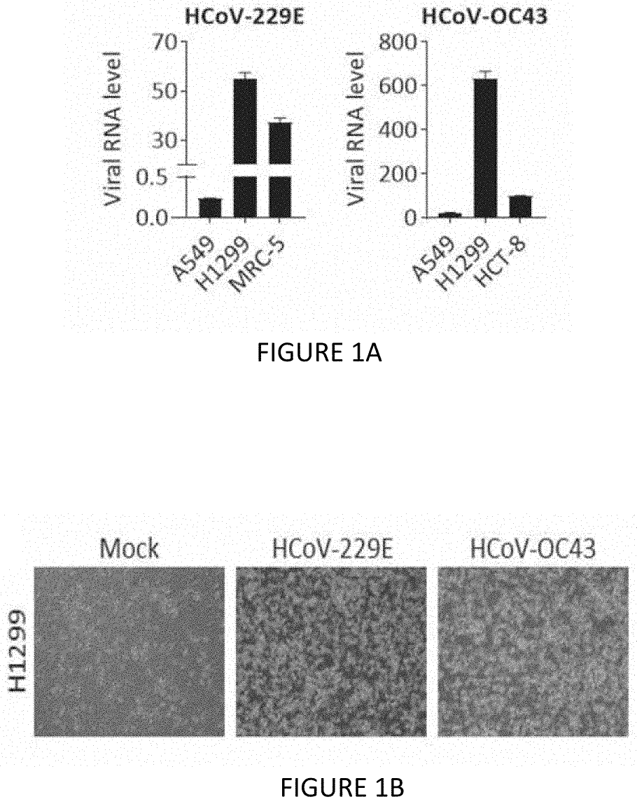

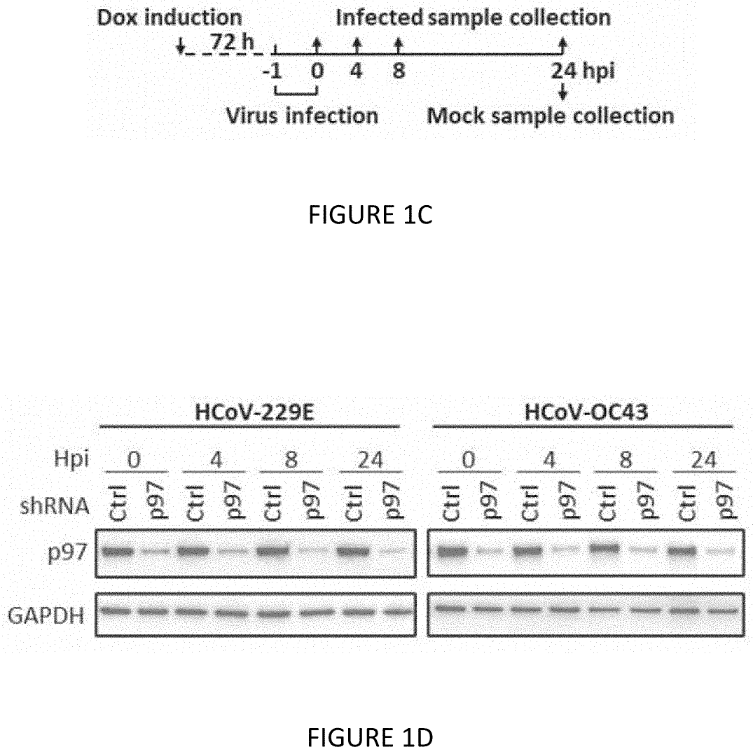

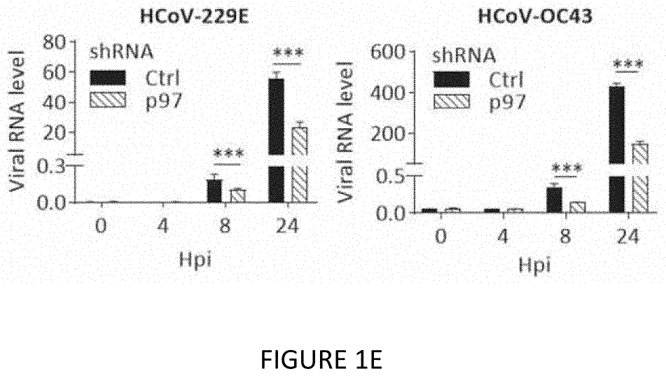

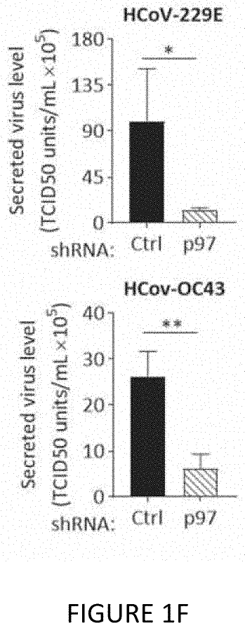

To identify cell lines that can be infected by both HCoV-229E and HCoV-OC43 viruses, two lung cancer cell lines, A549 and H1299, as well as the two virus propagating cell lines, MRC-5 and HCT-8 were tested. At 24 h post infection (hpi), cells were harvested to determine viral RNA levels. As shown in FIG. 1A, a higher viral RNA was detected in H1299 than in A549 cells (230- and 30-fold for HCoV-229E and HCoV-OC43, respectively; FIG. 1A). Extensive cytopathic effects (CPEs) were observed when H1299 cells were infected with either HCoV-229E or HCoV-OC43 at 48 hpi (FIG. 1B), indicating that viral infection progresses rapidly and robustly in infected H1299 cells. Therefore, the permissive H1299 cell line was selected as a model for both HCoV-229E and HCoV-OC43 infection in subsequent experiments.

To characterize the role of p97 in HCoV replication, p97 protein expression was knocked down using shRNA. p97 depletion may have pleiotropic effects in cells and be lethal given its essential roles in multiple cellular processes (Van den Boom, 2018). In addition, the abundance of p97 means that it takes several days to fully remove its function. Therefore, an inducible knockdown system to abolish p97 function was used. Stably transfected cells with Tet-regulated expression of p97-specific shRNA, or a control shRNA, using lentiviral infection was established. Immunoblot analysis confirmed that p97 expression was reduced by 90% in the presence of 0.5 mg/mL Dox for 72 h. Cells with control shRNA or p97 shRNA were induced using Dox for 72 h and followed by HCoV-229E, HCoV-OC43, or mock infection. Cells were harvested at 4 time points after HCoV infection (0, 4, 8, or 24 hpi) before detectable CPEs occur or after mock infection (24 hpi) (FIG. 1C). Efficient p97 depletion was efficient, as confirmed by the low expression levels observed across the infection time course (FIG. 1D). Viral RNA levels in HCoV-infected cells were measured. Both HCoV-229E and HCoV-OC43 RNA levels were detectable starting at 8 hpi and increased dramatically at 24 hpi (FIG. 1E). p97-knockdown cells displayed significantly lower viral RNA levels than controls: HCoV-229E and HCoV-OC43 RNAs were present at 58% and 66%, respectively, relative to controls at 24 hpi (FIG. 1E), suggesting that p97 is important for HCoV replication. Next, to examine the effect of p97 on the ability to produce progeny virions from HCoV-infected cells, viral titer in the culture media was quantified at 24 hpi by determining TCID50. p97 depletion resulted in a significant reduction in secreted viral titer by 87% for HCoV-229 and 76% for HCoV-OC43 (FIG. 1F). Taken together, w a functional cell model that is able to undergo a full replication cycle after HCoV infection was established. This model demonstrates that p97 is required for HCoV replication.

### Example 2: Similar Cellular Pathways are Induced by Infection with Different Coronaviruses

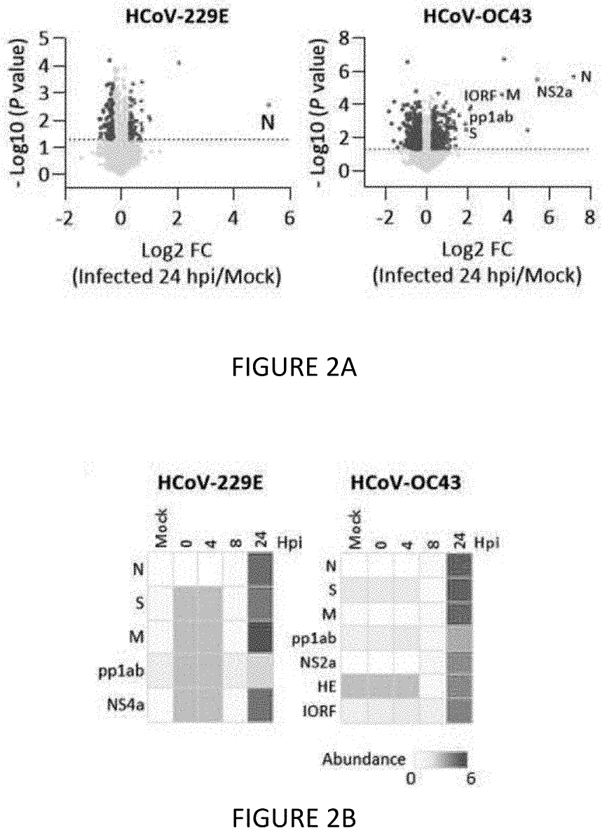

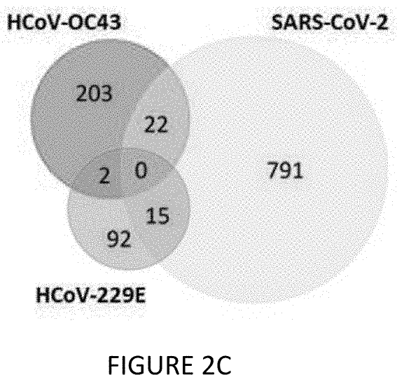

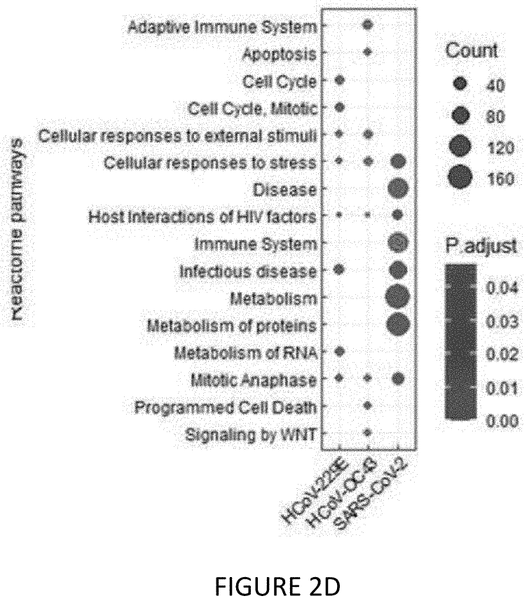

To test whether p97 is hijacked by HCoV during replication, proteomic analysis of cells treated with p97 or control shRNA after HCoV-229, HCoV-OC43, or mock infection was performed (FIG. 1C). In total, 6858 proteins across all replicates in the HCoV-229E infected cells were identified, of which five were HCoV-229E viral proteins. 7984 proteins were identified in the HCoV-OC43 infected cells, including seven HCoV-OC43 viral proteins. To understand the effects of HCoV on cells, changes in the proteome between HCoV-infected and mock-infected H1299 cells with control shRNA were analyzed. As shown in FIG. 2A, 147 differentially expressed proteins (DEPs) were identified (|log2 FC|>0.3, P<0.05) after HCoV-229E infection, of which 61 were up-regulated and 86 were down-regulated. HCoV-229E nucleoprotein (N) was significantly increased (FIG. 2A). In contrast, 609 DEPs (|log2 FC|>0.3, P<0.05) were obtained after HCoV-OC43 infection, with 288 up-regulated and 321 down-regulated DEPs (FIG. 2A). Of the increased DEPs, HCoV-OC43 viral proteins were significantly upregulated, including nucleoprotein (N), spike glycoprotein (S), membrane protein (M), non-structural protein 2a (NS2a), replicase polyprotein 1ab (pp1ab), and protein I (IORF) (FIG. 2A). Increased viral protein abundance during a 24-h period also confirmed viral infection (FIG. 2B). These results show that HCoV-229E and HCoV-OC43 successfully infect H1299 cells, and that HCoV-OC43 infection results in a greater change in the host proteome.

The effects of HCoV infection on cells were investigated and whether HCoV-229E, HCoV-OC43, and SARS-CoV-2 induce similar changes in cellular processes in infected cells were then tested. Comparison of shifts in pathways in response to virus infection in H1299 cells treated with control shRNAs was performed. In addition to HCoV-229 and HCoV-OC43, proteome results from SARS-CoV-2 infected Caco-2 cells were included (Bojkova, 2020). Hierarchical clustering was employed to identify proteins that increased over time after virus infection and 109, 227, and 828 proteins in the HCoV-229E, HCoV-OC43, and SARS-CoV-2 cluster were identified respectively. Overlap analysis showed no overlapping proteins across all the clusters (FIG. 2C). 15 proteins overlapping in HCoV-229E/SARS-CoV-2 and 22 proteins overlapping in HCoV-OC43/SARS-CoV-2 was obserned. Only 2 proteins overlapping in the proteome shifts observed upon HCoV-229E/HCoV-OC43 infection (FIG. 2C). Nevertheless, individual pathway enrichment analysis reveals that proteins in each cluster were enriched in the same pathways, including cellular responses to stress, host interactions of HIV factors, and mitotic anaphase (FIG. 2D). This pathway overlap indicates which host machineries are required for HCoV replication. Taken together, analysis of proteome changes after HCoV infection shows that common host pathways respond to HCoV-229E, HCoV-OC43, and SARS-CoV-2 infection, and may thus support HCoV replication.

### Example 3: Effects of p97 on Cell Cycle Pathways After HCoV Infection

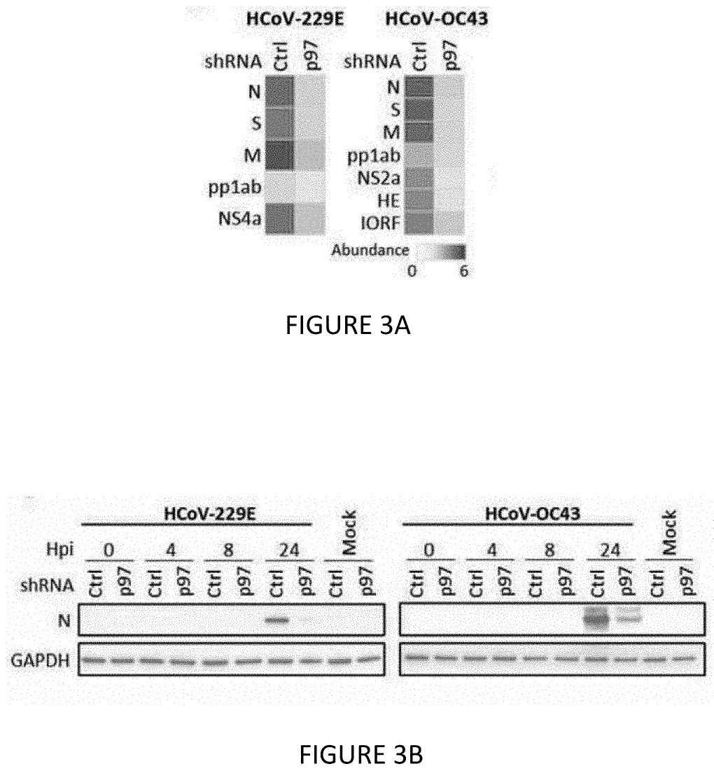

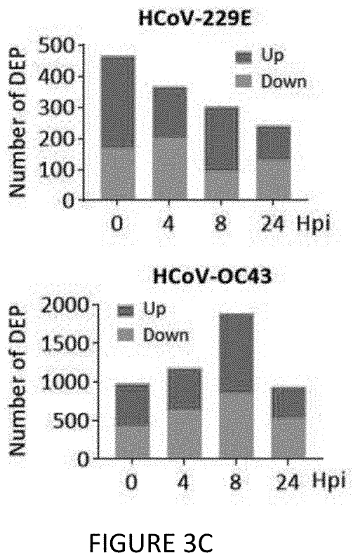

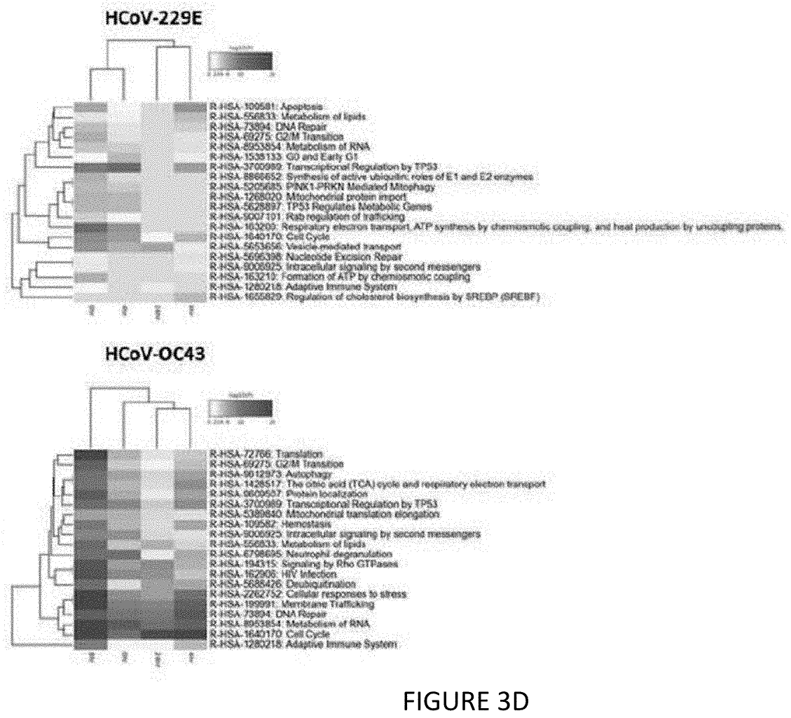

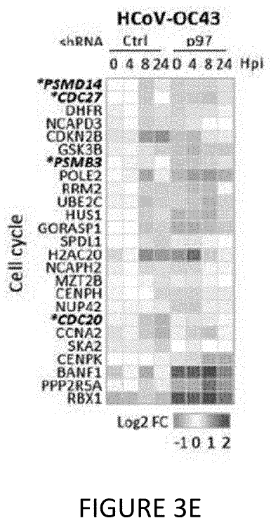

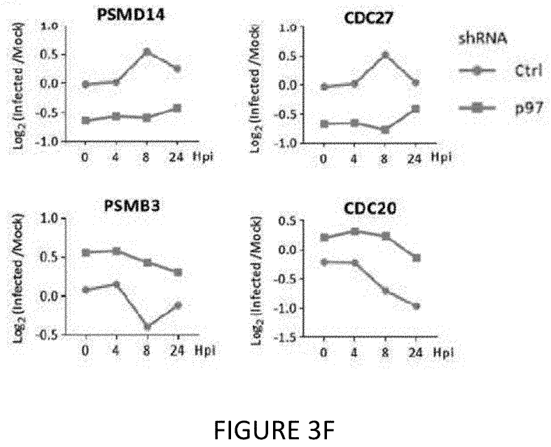

To determine the impact of p97 depletion on host responses during virus replication, viral protein abundance in cells was compared. Lower levels of viral proteins were observed in the infected p97-knockdown cells (FIG. 3A). The cellular levels of viral N protein over time after infection was also confirmed by immunoblot analysis. FIG. 3B shows that viral N proteins were detected in HCoV-229E or HCoV-OC43-infected cells at 24 hpi. Moreover, a reduction in viral N protein levels was observed in cells depleted of p97 at 24 hpi (FIG. 3B). Consistent with the above results, this data shows that p97 depletion reduced the synthesis of viral proteins in cells after infection, suggesting a pivotal role for p97 in both HCoV-229E and HCoV-OC43 replication.

Global changes in the host proteome over time between cells treated with p97 vs. control shRNA after HCoV infection were examined. Protein abundance in infected cells was normalized to that in corresponding mock-infected cells. Normalized protein levels between cells with control shRNA and p97 shRNA at each time point were analyzed. FIG. 3C shows the number of DEPs (|log2 FC|>0.3, P<0.05) over the infection time course for HCoV-229E or HCoV-OC43. Noticeably, HCoV-OC43 caused a shift in the proteome at 8 hpi, with 1047 up-regulated and 842 down-regulated DEPs identified (FIG. 3C). To uncover the functional effect of host responses during infection, multi-list pathway enrichment analysis of the DEPs was performed across the infection time course. As shown in FIG. 3D, proteins related to cell cycle were enriched across the whole period for both HCoV-229E or HCoV-OC43. The cell cycle-related category is a common change in the host proteome after HCoV-229E, HCoV-OC43, or SARS-CoV-2 infection (FIG. 2D). Therefore, changes in cell cycle proteins in cells treated with control and p97 shRNAs after HCoV-229E or HCoV-OC43 infection were examined. Twenty-five DEPs were identified across different time points distinct expression trends between the two cells after HCoV-OC43 infection (FIG. 3E). These DEPs include two proteasome related proteins, PSMD14 and PSMB3, and two components of the anaphase promoting complex or cyclosome (APC/C), CDC27 and CDC20. The APC/C and proteasome are two main regulators of the cell cycle (Castro, 2005; Reed, 2006). Interestingly, the dynamic levels of PSMD14, PSMB3, and CDC27 were different in cells treated with control shRNA compared to p97 shRNA after infection (FIG. 3F). While PSMD14 and CDC27 peaked in the infected cells with control shRNA at 8 hpi, no obvious changes were observed in p97-knockdown cells after HCoV-OC43 infection (FIG. 3F). Conversely, there was a decline in PDMB3 in cells with control shRNA at 8 hpi whereas p97 depletion led to a slow decrease in PDMB3 after infection (FIG. 3F). This suggests that p97 depletion may affect proteasome and APC/C function during HCoV replication. Collectively, these data show that multiple p97-dependent host processes support HCoV infection. When p97 is knocked down, these effects are abolished. These results suggest that p97 may be exploited to regulate the cell cycle through proteasome and the APC/C, thus facilitating viral replication.

### Example 4: p97 is Required for Early Stages of HCoV Replication

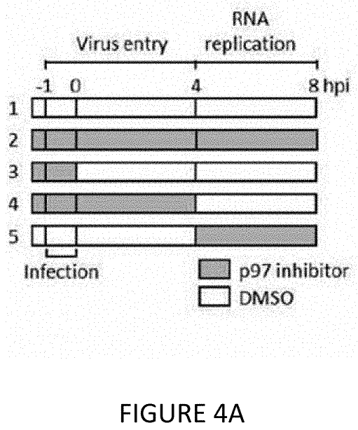

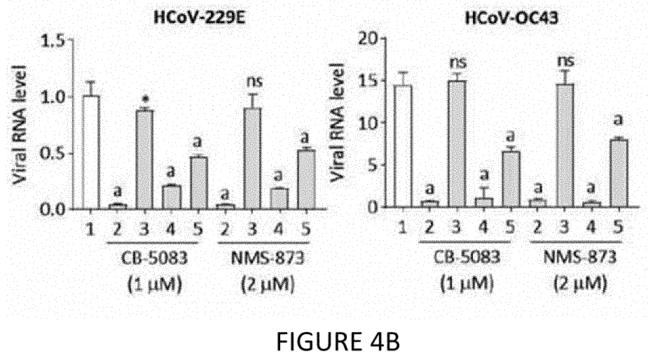

To verify the effects of p97 on different stages of HCoV replication cycle, two p97 inhibitors were used to perform a stage-limited inhibition assay. Based on the dynamics of viral RNA levels (FIG. 1E), it was determined that virus entry occurs at −1 to 4 hpi and RNA replication occurs at 4 to 8 hpi. Thus, cells were treated with vehicle or two p97 inhibitors, CB-5083 or NMS-873, at different timepoints post infection, and then harvested at 8 hpi to analyze viral RNA levels (FIG. 4A). Extensive reduction in viral RNA levels was observed in cells incubated with inhibitors throughout the experiment (group2) in both HCoV-229E and HCoV-OC43 infected cells compared to the vehicle treated control (group 1) (FIG. 4B). Importantly, early treatment (FIG. 4B, group 3: pretreatment to 0 hpi) caused only minor or no effects on viral RNA levels. However, significant reductions in viral RNA levels were observed in cells treated with inhibitors from pretreatment to 4 hpi (FIG. 4B, group 4), in which viral RNA levels were reduced by 79-82% and 92%-96% for HCoV-229E and HCoV-OC43 infection, respectively, suggesting that p97 is essential for HCoV entry but may not be important for early stages of viral infection such as receptor binding (FIG. 4B). Moreover, p97 inhibition at 4 to 8 hpi (FIG. 4B, group 5) also resulted in decreased viral RNA levels by 54-48% and 53-45% for HCoV-229E and HCoV-OC43 infection, respectively (FIG. 4B), indicating that p97 is also involved in processes linked to viral RNA replication. Altogether, these results confirm that p97 plays important functions at early stages of the virus life cycle, including in virus entry and viral RNA replication.

### Example 5: p97 is a Potential Therapeutic Target for HCoV Infection

To evaluate whether p97 is an effective therapeutic target for treating HCoV infection, the effect of the p97 inhibitor, CB-5083 was tested on HCoV replication in H1299 cells. H1299 cells were infected with HCoV-229E or HCoV-OC43 in the presence of different doses of CB-5083. Given the early effects on the proteome and infection seen above, cells were inoculated in media containing CB-5083 until 8 hpi. Cells were then washed and cultured in fresh media lacking the inhibitor. To investigate whether CB-5083 inhibits HCoV infection, cells were stained for the expression of double-stranded RNA (dsRNA) at 8 hpi, a hallmark of viral infection (V'kovski, 2021). As shown in FIG. 5A, lower dsRNA signals were observed in the presence of CB-5083 (0.5 mM) after infection with HCoV-229E or HCoV-OC43, indicating p97 inhibition is able to suppress HCoV infection. The effects of CB-5083 on progeny virus production were then examined.

Cellular viral RNA and secreted virus was determined in the samples harvested at 24 hpi. HCoV-229E and HCoV-OC43 viral RNA level was reduced by CB-5083 in a dose-dependent manner (FIG. 5B). Notably, CB-5083 at 1 mM significantly reduced levels of HCoV-229E and HCoV-OC43 viral RNA by about 47% and about 40% (FIG. 5B). In addition, titer of progeny virus in the cultured media was quantified. FIG. 5C, shows that CB-5083 reduced progeny virus levels. The viral titer of secreted HCoV-229E and HCoV-OC43 was significantly decreased by ˜81% and ˜85% in the presence of 1 mM CB-5083. Taken together, these results confirm that p97 inhibition has antiviral activity during HCoV infection.

Capability of CB-5083 in preventing CPEs caused by HCoV infection was tested. H1299 cells were infected with HCoV-229E, HCoV-OC43, or mock-treated in the presence of different concentrations of CB-5083. Cell viability were examined at 72 hpi. The results for each infection was normalized to the mock-infected and DMSO-treated control cells. The tested concentrations of CB-5083 were non-toxic to H1299 cells (FIG. 5D). Notably, CB-5083 (0.5 mM) significantly increased cell viability by ˜29% compared to the HCoV-infected and DMSO-treated control, indicating that p97 inhibition protects cells from HCoV infection. Collectively, the data show the potential of p97 as the therapeutic target for HCoV treatment.

In at least some of the previously described embodiments, one or more elements used in an embodiment can interchangeably be used in another embodiment unless such a replacement is not technically feasible. It will be appreciated by those skilled in the art that various other omissions, additions, and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “ a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one of skill in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those of skill in the art. The various aspects and embodiments disclosed herein are for purposes of illustration and are not intended to be limiting, with the true scope and spirit being indicated by the following claims.

### REFERENCES

Ballar P, Fang S. 2008, Regulation of ER-associated degradation via p97/VCP-interacting motif. Biochem Soc Trans 36:818-822.

Bojkova D, Klann K, Koch B, Widera M, Krause D, Ciesek S. Cinatl J, Munch C. 2020. Proteomics of SARS-CoV-2-infected host cells reveals therapy targets. Nature 583:469-472.

Bouhaddou M, Memon D, Meyer B, White K M, Rezelj V V, Correa Marrero M, Polacco B J, Melnyk J E, Ulferts S, Kaake R M, Batra J, Richards A L, Stevenson E, Gordon D E, Roje A, Obernier K, Fabius J M, Soucheray M, Miorin L, Moreno E, Koh C, Tran Q D, Hardy A, Robinot R, Vallet T, Nilsson-Payant B E, Hernandez-Armenta C, Dunham A, Weigang S, Knerr J, Modak M, Quintero D, Zhou Y. Dugourd A, Valdeolivas A, Patil T, Q, Hüttenhain R, Cakir M, Muralidharan M, Kim M, Jang B, Tutuncuoglu B, Hiatt J, Guo J Z, Xu J, Bouhaddou S, Mathy C J P, Gaulton A, Manners E J, Felix E, Shi Y, Goff M, Lim J K, McBride T, O'Neal M C, Cai Y, Chang J C J, Broadhurst D J, Kiippsten S, De wit E, Leach A R, Kortemme T, Shoichet B, Ott M, Saez-Rodriguez J, tenOever B R, Mullins R D, Fischer E R, Kochs G, Grosse R, Garcia-Sastre A, Vignuzzi M, Johnson J R, Shokat K M, Swaney D L, Beltrao P, Krogan N J. 2020, The Global Phosphorylation Landscape of SAPS-CoV-2 Infection. Cell 182:685-712.e19.

Castro A, Bernis C, Vigneron S, Labbé J-C, Lorca T. 2005. The anaphase-promoting complex: a key factor in the regulation of cell cycle. 3. Oncogene 24:314-325.

Emergency Use Authorization (EUA) for remdesivir, an unapproved product Center for Drug Evaluation and Research (CDER) Review 37.

Fonseka P, Pathan M, Chitti S V, Kang T, Mathivanan S. 2021, FunRich enables enrichment analysis of OMICs datasets. J Mol Biol 433:166747.

Fung T S, Huang M, Liu D X. 2014. Coronavirus-induced ER stress response and its involvement in regulation of coronavirus—host interactions. Virus Research 194:110-123.

Hoffmann M, Kleine-Weber H, Schroeder S, Kruger N, Herrler T, Erichsen S, Schiergens T S, Herrler G, Wu N-H, Nitsche A, Müller M A, Drosten C, Pöhlmann S. 2020. SARS-CoV-2 Cell Entry Depends on ACE2 and TMPRSS2 and Is Blocked by a Clinically Proven Protease Inhibitor. Cell 181:271-280.e8.

Hofmann H, Pyrc K, van der Hoek L, Geier M, Berkhout B, Pöhlmann S. 2005. Human coronavirus NL63 employs the severe acute respiratory syndrome coronavirus receptor for cellular entry. Prot Nati Acad Sci USA 102:7988-7993.

Hu. B, Guo H, Zhou P, Shi Z-L. 2021. Characteristics of SARS-CoV-2 and COVID-19. Nat Rev Microbiol 19:141-154.

Jassal B, Matthews L, Viteri G, Gong C, Lorente P, Fabregat A, Sidiropoulos K, Cook J, Gillespie M, Haw R, Loney F, May B, Iilat is M, Rothfels K, Sevilla. C, Shamovsky V, Shorser S, Varusai T, Weiser J, G, Stein L, Hermjakob H, D'Eustachio P. 2019. The reactome pathway knowledgebase. Nucleic Acids Research gkz1031.

Kokic G, Hillen H S, Tegunov D, Dienemann C, Seitz F, Schmitzova J, Farming L, Siewert A, Höbartner C, Cramer P. 2021. Mechanism of SARS-CoV-2 polymerase stalling by remdesivir. Nat Common 12:279.

Li W, Moore M J, Vasilieva N, Sui J, Wong S K, Berne M A, Somasundaran M, Sullivan J L, Luzuriaga K, Greenough T C, Choe H, Farzan M. 2003. Angiotensin-converting enzyme 2 is a functional receptor for the SARS coronavirus. 6965. Nature 426:450-454.

Liu D X, Liang J Q, Fung T S. 2021. Human Coronavirus-229E, -OC43, -NL63, and -HKU1 (Coronaviridae), p. 428-440. In Encyclopedia of Virology. Elsevier.

Papadopoulos C, Kirchner P, Bug M, Grum D, Koerver L, Schulze N, Poehler R, Dressler A, Fengler S, Arhzaouy K, Lux V, Ehrmann M, Weihl C C, Meyer H. 2017. VCP/p97 cooperates with YOD1, UBXD1 and PLAA to drive clearance of ruptured lysosomes by autophagy. The EMBO Journal 36:135-150.

Parisi E, Yahya G, Flores A, Aldea. M, 2018. Cdc48/p97 segregase is modulated by cyclin-dependent kinase to determine cyclin fate during GI progression. The EMBO Journal 37:e98724.

Pathan M, Keerthikumar S, Ang C-S, Gangoda L, Quek C Y J, Williamson N A, Mouradov D, Sieber O M, Simpson R J, Salim A, Bacic A, Hill A F, Stroud D A, Ryan M T, Agbinya J I, Mariadason J M, Burgess A W, Mathivanan S. 2015. Fun Rich: An open access standalone functional enrichment and interaction network analysis tool. PROTEOMICS 15:2597-2601.

Pathan M, Keerthikumar S, Chisanga D, Alessandro R, Ang C-S, Askenase P, Batagov A. O. Benito-Martin A, Camussi G, Clayton A, Collino F, Di Vizio D, Falcon-Perez J M, Fonseca P, Fonseka P, Fontana S, Gho Y S, Hendrix A, Hoen EN-'t, Traci N, Kastaniegaard K, Kislinger T, Kowal J, Kurochkin I V, Leonardi T, Liang Y, Llorente A, Lunavat T R, Maji S, Monteleone F, Øverbye A, Panaretakis T, Patel T, Peinado H, Pluchino S, Principe S, Ronquist G, Royo F, Sahoo S, Spinelli C, Stensballe A, Théry C, van Herwijnen M J C, Wauhen M, Welton J L, Zhao K, Mathivanan S. 2017. A novel community driven software for functional enrichment analysis of extracellular vesicles data. J Extracell Vesicles 6.

Radhakrishnan S K, den Besten W, Deshaies R J. 2014. p97-dependent retrotranslocation and proteolytic processing govern formation of active Nrf1 upon proteasome inhibition. eLife 3:e01856.

Raj V S, Mou H, Smits S L, Dekkers D H W, Müller M A, Dijkman R, Muth D, Demmers J A A, Zaki A, Fouchier R A M, Thiel V, Drosten C, Rottier P J M, Osterhaus A D M E, Bosch B J, Haagmans B L. 2013. Dipeptidyl peptidase 4 is a functional receptor for the emerging human coronavirus-EMC. 7440. Nature 495:251-254.

Raudvere U, Kolberg L, Kuzmin I, Arak T, Adler P, Peterson H, Vilo J. g:Profiler: a web server for functional enrichment analysis and conversions of gene lists (2019 update) 8.

Reed S I. 2006. The ubiquitin-proteasome pathway in cell cycle control. Results Probl Cell Differ 42:147-181.

Shang J, Wan Y, Luo C, Ye G, Geng Q, Auerbach A, Li F. 2020, Cell entry mechanisms of SARS-CoV-2. PNAS 117:11727-11734.

Su M, Chen Y, Qi S. Shi D, Feng L, Sun D. 2020. A Mini-Review on Cell Cycle Regulation of Coronavirus Infection. Front Vet Sci 7.

van den Boom J. Meyer H. 2018. VCP/p97-Mediated Unfolding as a Principle in Protein Homeostasis and Signaling. Molecular Cell 69:182-194.

V'kovski P, Kratzel A, Steiner S, Stalder H, Thiel V. 2021. Coronavirus biology and replication: implications for SARS-CoV-2. Nat Rev Microbiol 19:155-170.

Yang N, Shen H-M. 2020. Targeting the Endocytic Pathway and Autophagy Process as a Novel Therapeutic Strategy in COVID-19. Int J Biol Sci 16:1724-1731.

Yeager C L, Ashmun R A, Williams R K, Cardellichio C B, Shapiro L H, Look A T, Holmes K V. 1992. Human aminopeptidase N is a receptor for human coronavirus 229E. Nature 357:420-422.

Zhou Y, Zhou B, Pache L, Chang M, Khodabakhshi A H, Tanaseichuk O, Benner C, Chanda S K. 2019. Metascape provides a biologist-oriented resource for the analysis of systems-level datasets. Nat Commun 10:1523.

What is claimed is:

1. A method of reducing, improving, or treating a viral infection, the method comprising:
identifying a subject having a viral infection, or a symptom thereof; and
administering an effective amount of an agent that promotes inhibition of p97 in the subject,
wherein the viral infection or a symptom thereof is reduced after the administering.

2. The method of claim 1, wherein the viral infection is a coronavirus infection.

3. The method of claim 2, wherein the coronavirus infection is caused by the members of the Coronavirinae subfamily of viruses.

4. The method of claim 3, wherein the members of Coronavirinae subfamily of viruses include HCoV-NL63, HCoV-HKU1, HCoV-229E, HCoV-OC43, severe acute respiratory syndrome coronavirus (SARS-CoV), severe acute respiratory syndrome coronavirus 2 (SARS-CoV-2) or MERS-CoV.

5. The method of claim 1, wherein the agent that promotes inhibition of p97 is an inhibitory nucleic acid molecule, p97 binding antagonist, a genetic tool, and/or a small molecule inhibitor.

6. The method of claim 1, wherein the subject having a coronavirus infection expresses or synthesizes one or more proteins involved in the cell cycle pathway differently than in normal subjects.

7. The method of claim 6, wherein the proteins involved in the cell cycle pathway comprise components of the proteasome and the anaphase promoting complex or cyclosome (APC/C).

8. The method of claim 7, wherein the components of the proteasome and the APC/C comprise PSMD14, PSMB3, CDC27 and CDC20.

9. The method of claim 5, wherein the inhibitory nucleic acid molecule is an antisense nucleic acid.

10. The method of claim 5, wherein the inhibitory nucleic acid molecule is a siRNA.

11. The method of claim 5, wherein the inhibitory nucleic acid molecule is a shRNA.

12. The method of claim 5, wherein the inhibitory nucleic acid molecule corresponds to or is complementary to at least a fragment of nucleic acid encoding p97.

13. The method of claim 5, wherein the p97 binding antagonist inhibits the binding of p97 to its binding partners.

14. The method of claim 13, wherein the p97 binding antagonist is an antibody against p97 or a fragment of p97.

15. The method of claim 14, wherein the antibody is a monoclonal, polyclonal or an antibody fragment selected from the group consisting of Fab, Fab′-SH, Fv, scFv, and (Fab′)₂ fragments.

16. The method of claim 5, wherein the genetic tool is selected from the group consisting of a CRISPR/Cas9 system, a zinc finger nuclease system, a TALEN system, a homing endonucleases system or a meganuclease system.

17. The method of claim 5, wherein the small molecule inhibitor is CB-5083, NMS-873, NMS-859, DBeQ, MSC1094308, ML240, p97-IN-1, VCP/p97 inhibitor-1, ML241 hydrochloride, or UPCDC-30245.

18. The method of claim 1, wherein the inhibition of p97 in the subject reduces or decreases the expression or levels of viral proteins in the subject.

19. The method of claim 18, wherein the viral proteins comprise nucleoprotein (N), spike glycoprotein (S), membrane protein (M), non-structural protein 2a (NS2a), non-structural protein 4a (NS4a), replicase polyprotein 1ab (pp1ab) and protein 1 (1ORF).

20. The method of claim 1, wherein the inhibition of p97 in the subject reduces or decreases the viral titer of coronavirus.

21. The method of claim 1, wherein the inhibition of p97 in the subject reduces the cytopathic effects caused by the coronavirus infection.

22. The method of claim 1, wherein the inhibition of p97 in the subject reduces or suppresses the replication of coronavirus.

23. A method of identifying a subject having a coronavirus infection, the method comprising detecting at least one of:
a level of a product or expression of a gene of the subject selected from the group consisting of: CDC20, CDC27, PSMD14 and PSMB3, or a combination of two or more of the listed genes.

24. The method of claim 23, wherein detecting a level of product or gene expressed differently in normal and subjects with coronavirus infection.

25. A method of reducing, improving, or treating a coronavirus infection, the method comprising:
detecting the level, and/or expression of at least one or more of CDC20, CDC27, PSMD14 and PSMB3 in a subject;
comparing the level, and/or expression of one or more of CDC20, CDC27, PSMD14 and PSMB3 in the subject to the level and/or expression of one or more of CDC20, CDC27, PSMD14 and PSMB3 in the normal subject, wherein detection of an abnormal level and/or expression of one or more of CDC20, CDC27, PSMD14 and PSMB3 in the subject relative to the normal subject indicates the presence of a coronavirus infection in the subject; and
administering to the subject an effective amount of an agent that promotes inhibition of p97 in the subject, wherein the agent that promotes inhibition of p97 is selected from the group consisting of an inhibitory nucleic acid molecule, p97 binding antagonist, a genetic tool, and/or a small molecule inhibitor;
wherein the coronavirus infection or a symptom thereof is reduced after the administering.

26. The method of claim 25, wherein the coronavirus infection is caused by the members of the Coronavirinae subfamily of viruses.

27. The method of claim 26, wherein the members of Coronavirinae subfamily of viruses include HCoV-NL63, HCoV-HKU1, HCoV-229E, HCoV-OC43, severe acute respiratory syndrome coronavirus (SARS-CoV), severe acute respiratory syndrome coronavirus 2 (SARS-CoV-2) or MERS-CoV.

28. The method of claim 25, wherein the agent that promotes inhibition of p97 is an inhibitory nucleic acid molecule, p97 binding antagonist, a genetic tool, and/or a small molecule inhibitor.

29. The method of claim 25, wherein the subject having a coronavirus infection expresses or synthesizes one or more proteins involved in the cell cycle pathway differently than in normal subjects.

30. The method of claim 25, wherein the inhibitory nucleic acid molecule is an antisense nucleic acid.

31. The method of claim 25, wherein the inhibitory nucleic acid molecule is a siRNA.

32. The method of claim 25, wherein the inhibitory nucleic acid molecule is a shRNA.

33. The method of claim 25, wherein the inhibitory nucleic acid molecule corresponds to or is complementary to at least a fragment of nucleic acid encoding p97.

34. The method of claim 25, wherein the p97 binding antagonist inhibits the binding of p97 to its binding partners.

35. The method of claim 34, wherein the p97 binding antagonist is an antibody against p97 or a fragment of p97.

36. The method of claim 35, wherein the antibody is a monoclonal, polyclonal or an antibody fragment selected from the group consisting of Fab, Fab′-SH, Fv, scFv, and (Fab′)2 fragments.

37. The method of claim 25, wherein the genetic tool is selected from the group consisting of a CRISPR/Cas9 system, a zinc finger nuclease system, a TALEN system, a homing endonucleases system or a meganuclease system.

38. The method of claim 25, wherein the small molecule inhibitor is CB-5083, NMS-873, NMS-859, DBeQ, MSC1094308, ML240, p97-IN-1, VCP/p97 inhibitor-1, ML241 hydrochloride, or UPCDC-30245.


SEQUENCE LISTING (1 sequence)

SEQ ID NO: 1 - DNA, 22 bp, Artificial Sequence
  Nucleic acid
aaacagccattctcaaacagaa